(19) United States
(12) Patent Application Publication
(10) Pub. No.: US 2020/0035337 A1
(43) Pub. Date: Jan. 30, 2020

(54) METHOD AND PRODUCT FOR DETERMINING A STATE VALUE, A VALUE REPRESENTING THE STATE OF A SUBJECT

(71) Applicant: FOLLOWFLOW HOLDING B.V., Venlo (NL)

(72) Inventors: Jurjen SÖHNE, Amsterdam (NL); Fabian Harmen TIJSSEN, Meessen (NL); Anthonius Albertus Johannes DE GRAAFF, Venlo-Blerick (NL)

(21) Appl. No.: 15/735,883

(22) PCT Filed: Jun. 9, 2016
(86) PCT No.: PCT/EP2016/000952

§ 371 Date: Dec. 12, 2017

(30) Foreign Application Priority Data
Jun. 17, 2015 (NL) 1041367
Oct. 6, 2015 (NL) 1041503
Oct. 28, 2015 (NL) 1041550
Nov. 5, 2015 (NL) 1041555
Nov. 19, 2015 (NL) 1041580
Dec. 2, 2015 (NL) 1041606
Dec. 17, 2015 (NL) 1041629
Jan. 22, 2016 (NL) 1041686
Feb. 3, 2016 (NL) 1041708

(51) Int. Cl. G16H 10/60; G08B 7/00; G08B 21/18; A61B 5/024; A61B 5/00

(52) U.S. Cl. CPC G16H 10/60; G08B 7/00; G08B 21/182; A61B 5/02405; A61B 5/0022; A61B 5/7455; A61B 5/6898; A61B 5/02438; A61B 5/486; A61B 5/7405; A61B 5/742; A61B 5/681

(57) ABSTRACT

The present invention provides a method of calculating certain body and mental rhythms, and then using this information to indirectly determine the state (flow, body and mental rhythms) which is also reflective of balance between the sympathetic and parasympathetic portions of the autonomic nervous system of the user. By calculating the HRV real time including High Frequency components of the heart information, and for example determining the flow by multiplying the Heart Rate frequency with the Heart Rate Variability, a new method for determining a user state is invented. It is efficient and effective method to use less power within a faster response time for determining such a state. It is applicable in the frequency and time domain.

### TECHNICAL FIELD

The present invention relates generally to the determination, evaluation, feedback, etc. of a state of a user based upon heart rate variability and heart rate frequency. The present system, and method relates generally to a heart rhythm determination system and method applying this and particularly, but not by way of limitation, to such a system and method.

### BACKGROUND

In this paragraph the meaning of the word flow is introduced. In positive psychology, flow, also known as being in the zone, is the mental state of operation in which a person performing an activity is fully immersed in a feeling of energized focus, full involvement, and enjoyment in the process of the activity. In essence, flow is characterized by complete absorption in what one does. Named by Mihály Csíkszentmihályi, the concept has been widely referenced across a variety of fields, though has existed for thousands of years under other guises, notably in some eastern religions. Achieving flow is often referred to as being in the zone.

In this paragraph the meaning of the word Heart Rate Variability is introduced. Heart rate variability (HRV) is the physiological phenomenon of variation in the time interval between heartbeats. It is measured by the variation in the beat-to-beat interval. Reduced HRV has been shown to be a predictor of mortality. Currently, there are several smartphone and tablet apps that offer Heart Rate Variability readings. The current technology sometimes requires connection of these devices to a heart rate strap via Bluetooth Smart or other type of heart rate sensor. Though there are also apps which simply watch the varying, via for example color changes, of a heart pulse through one's fingertip, by for example the phone camera.

What is a “good” HRV? There's no universal answer to this, but we can take some clues from a 2010 American Journal Cardiology study by Zulfigar et al. at the U. Illinois Medical Center. They measured the HRV of 344 healthy subjects aged 10-99. Keep in mind that these HRV values were calculated using heart information data over full 24-hours. A plot from the Zulfigar study, in FIG. 3 shows the distribution of HRV values by age. It shows a lot of variation by age, but a clear trend towards lower HRV values as one gets older.

One interesting non-fitness benefit that can be noticed from measuring HRV is when there is a “high-stress” reading, it is possible to actively and subconsciously to calm and relax oneself. Dr. Ron Sinha, uses HRV monitoring in his patients with metabolic syndrome and covers the topic extensively in The South Asian Health Solution. Start by breathing more deeply, get a lid on the racing thoughts, and just generally slow down, by this the HRV values will increase. By instant feedback the effects of users response in real time can be used.

With the growing complexity of life, the relation between physiological conditions and emotional health becomes of increasing interest. Many studies have shown that stress and other emotional factors may increase the risk of disease, reduce performance and productivity and severely restrict the quality of life. To this end, the medical communities around the world continually seek remedies approaches and preventive action plans. Recently a focus on the self-regulation of systems within the body has led to research in the areas of biofeedback, etc.

A variety of new techniques have been introduced as alternatives to more traditional psychotherapies or pharmaceutical interventions for improving mental and/or emotional imbalances. In addition to the more psychological approaches like cognitive re-structuring and neuro-linguistic programming, psychologists have employed several techniques from Eastern cultures to “still the mind” during focused meditation.

Positive emotional states, generate changes in the dynamic beating patterns of the heart. A method for quantifying and analyzing these heart rhythms is called analysis of heart rate variability (HRV). The normal resting heart rate in healthy individuals varies dynamically from moment to moment. Heart rate variability, which is derived from the electro-cardiogram (ECG) or heart pulses, is a measure of these naturally occurring beat-to-beat changes in heart rate and is an important indicator of health and fitness. HRV is influenced by a variety of factors, wherein comprising physical movement, sleep and mental activity, and is particularly responsive to stress and changes in emotional state. The analysis of HRV can provide important information relative to the function and balance of the autonomic nervous system, as it can distinguish sympathetic from parasympathetic regulation of heart rate. Decreased HRV is also a powerful predictor of future heart disease, increased risk of sudden death, as well as all-cause mortality.

Individual's HRV pattern and respiration can be synchronized, as can happen spontaneously in states of deep relaxation, sleep or when using techniques to facilitate autonomic balance. In most individuals, small to near-zero HRV, as just described, is an indicator of a potentially pathological condition or aging because it connotes loss of flexibility of the heart to change in rate. However, in trained subjects, it is an indication of exceptional self-management of their emotions and autonomic nervous system because their HRV is normally large and the shift into the internal coherence mode is a result of intentionally entering the amplified peace state. This is very different from a pathological condition underlying lowered HRV (in such cases the HRV is always low).

Conscious focus of attention and/or positive emotions has been shown to significantly influence HRV.

When functioning properly, the human heart maintains its own intrinsic rhythm, and is capable of pumping adequate blood throughout the body's circulatory system.

However, some people have irregular cardiac rhythms, referred to as cardiac-arrhythmias. Such arrhythmias result in diminished blood circulation. One mode of treating cardiac arrhythmias uses drug therapy. Drugs are often effective at restoring normal heart rhythms. However, drug therapy is not always effective for treating arrhythmias of certain patients. For such patients, an alternative mode of treatment is needed. One such alternative mode of treatment includes the use of a cardiac rhythm management system. Such systems can be implanted in the patient and deliver therapy to the heart.

Heart rhythm management systems include, among other things, pacemakers, defibrillators, or monitoring systems in the form of wearables wherein applications on smart wearables (smart watches, smart phones).

Physicians treating patients, monitoring patients for providing a prognosis or monitoring athletes require valid measuring data for their analyses, or users just want to quantify themselves for adapting well-being during high stress work or for adjusting therapy or behavior.

Heart rate variability (HRV) is thought to provide one such assessment of cardiovascular health. The time interval between intrinsic ventricular heart contractions changes in response to the body's metabolic need for a change in heart rate and the amount of blood pumped through the circulatory system. For example, during a period of exercise or other mental stress activity, a person's intrinsic heart rate will generally increase over a time period of several or many heartbeats. However, even on a beat-to-beat basis, that is, from one heart beat to the next, and without exercise, the time interval between intrinsic heart contractions varies in a normal person. These beat-to-beat variations in intrinsic heart rate are the result of proper regulation by the autonomic nervous system of blood pressure and cardiac output; the absence of such variations indicates a possible deficiency in the regulation being provided by the autonomic nervous system.

The autonomic nervous system itself has two components: sympathetic and parasympathetic (or vagal). The sympathetic component of the autonomic nervous system is associated with a tendency to raise heart rate, blood pressure, and/or cardiac output. The parasympathetic/vagal component of the autonomic nervous system is associated with a tendency to reduce heart rate, blood pressure, and/or cardiac output.

A proper balance between the sympathetic and parasympathetic components of the autonomic nervous system is important.

Therefore, an indication of this balance of the components of the autonomic nervous system, which is sometimes referred to as “autonomic balance,” “sympathic tone,” or “sympathovagal balance,” provides a useful indication of the patient's well-being.

One technique for providing an indication of the balance of the components of the autonomic nervous system is provided by the beat-to-beat heart rate variability, as discussed above. More particularly, intrinsic ventricular contractions are detected. The time intervals between these contractions, referred to as the R-R intervals, are recorded after filtering out any ectopic contractions, that is, ventricular contractions that are not the result of a normal sinus rhythm. This signal of R-R intervals is typically transformed into the frequency-domain, such as by using Fast Fourier Transform (FFT) techniques, so that its spectral frequency components can be analyzed. Another method is that this signal of R-R intervals is used in the time-domain to calculate the time domain HRV.

A problem arises for both, the frequency-domain HRV methods and the time-domain HRV methods with respect to the defined standards to calculate HRV is that these methods are not real time. The standard calculation method of HRV is averaging the variable heart information too long and is not excluding certain averaged heart beat information. The calculated HRV standard proposes to average the heart rate variability over a longer period than for example 50 beats. Such an averaging in the calculation of HRV results in that short term effects of the HRV are not detectable in the calculated standard HRV. Therefore, there is a need to provide a method to calculate HRV which is real time and as a result provide faster and better (real time) variation heart beat information about the subject state such as an indication of user well-being.

There is a need to provide quantified information regarding the users' balance which is easily used and does not require extensive biofeedback equipment. Further there is a need for a mobile method of monitoring this balance for use in everyday life.

It is an objective of the invention to provide a method and system that is very fast and uses little power.

### DISCLOSURE OF INVENTION

In order to achieve this objective, according to the invention, a method, product, system, or network comprising the steps of;
receiving a heart beat information,
calculating a heart rate frequency value, wherein the heart rate frequency value is a number of heart beats per first time of the heart beat information,
calculating a heart rate variability value, wherein the heart rate variability value is a variation time per second time of the heart beat information,
determining the state value from the heart rate frequency value and the heart rate variability value by using one of multiplication, addition, or other type of relationship, wherein the first time and second time is one minute or less.

By doing so the present invention provides a method of determining the state value in a detailing specific, a concise and fast way, beat to beat including all heart beat information in two state values being HR and real time HRV.

According to one aspect of the present invention, the embodiment further comprises;
calculating the first heart rate variability value for 30 or less heart beats of the heart beat information, wherein the first heart rate variability value is an absolute difference between a mean RR time between these heart beats and a RR time between heart beats,
determining the state value from the heart rate variability value.

By doing so the present invention provides a method of determining the state value with a HRV real time method which is fast and energy efficient.

According to one aspect of the present invention, the embodiment further comprises;
calculating a second heart rate variability value for 60 or less heart beats, wherein the second heart rate variability value is calculated by taking the average of the calculated first heart rate variability values.

By doing so the present invention provides a method of calculating Heart Rate Variability very fast, and includes all (without removing) high frequency information of the heart beat information and is real time. By doing this way the heart rate variability (value) is separated fully from the mean pp heart rate behavior. The mean pp heart rate behavior is represented by heart rate frequency value. By doing it in this way is possible to measure real time certain body and mental rhythms real time represented by heart rate variability value and heart rate frequency value, and then using this information to indirectly determine the (for example flow, physical and/or emotional) state (body and mental rhythms) which is also reflected in the balance between the sympathetic and parasympathetic portions of the autonomic nervous system of the user.

Measuring of the heart beat information is done in the time domain. Heart rate frequency value, and/or heart rate variability value can be calculated in time or frequency domain. Part of this invention is the way the heart rate variability is calculated which results in that the heart rate variability value represents the stroke volume variability of the heart beat information and this is the high frequency of the heart beat information per heart beat. By calculating the HRV as an average of short term HRV values a more stable HRV state can be determined.

According to one aspect of the present invention, the embodiment further comprises;
determining the state value in a real time manner per heartbeat.

By doing so the present invention provides a method of determining the state value per heart beat in a fast and precise matter which results in really fast state determination method.

According to one aspect of the present invention, the embodiment further comprises;
determining the state value in a real time manner per heartbeat per information source,
logging per information source the state value.

By doing so for example via big data a filtering mechanism can be provided (while browsing) to the user for only showing information to the user that will increase flow.

According to one aspect of the present invention, the embodiment further comprises;
determining the state value in a real time manner per heartbeat,
determining an accuracy level of the heart information.

By doing so the user is for example only getting heart information when an certain accuracy is maintained during the heart information measurement or for example is switched to a more accurate heart sensor in case of low accuracy.

According to one aspect of the present invention, the embodiment further comprises;
determining the state value in a real time manner per heartbeat,
determining a local maximum value of the state value after which the state value decreases.

By doing so the user state status can be easily determined during a dynamic flow training.

Above methods are implemented as a program code on a computer readable media, and such program code is loaded into a computer, by which computer is behaving as method of claim 1. Several device types can be used for such a method. Examples are smart watch, smart phone, server machine, or cloud machine, which are behaving as one of the methods of claim 1.

By for example multiplying (in time domain) the Heart Rate value with the Heart Rate Variability value a new method for determining a flow user state is invented (in frequency domain for example proposed is to use addition). It is an efficient and effective method to use less power within a faster response time for determining such a state.

An advantage of such method for determining a (for example flow) state value, a value representing the (for example happiness) state of a subject, is that the state value is scaling easily and correctly with the mental task or physical task of a user. Such a state value can be easily calculated and is power efficient and real-time calculated and the state value can be presented to the user.

The invention has been explained here-above in the example of a method, system, product, application, or network for determining the state (body and mental rhythms) which is also reflective in balance between the sympathetic and parasympathetic portions of the autonomic nervous system of the user.

The invention also relates to a software product comprising program code on a computer-readable medium, wherein said program code, when loaded into a computer that is connected to (for example a wearable) system (for example within a network) according to the invention causes the computer to act according to a method of the invention.

Preferred embodiments will now be described in conjunction with the drawings.

### BRIEF DESCRIPTION OF THE DRAWINGS

The present invention may be more fully understood by a description of certain preferred embodiments in conjunction with the attached drawings in which:

FIG. 1 illustrates user measured heart rate information data points represented by related and calculated heart rate variability values (HRV) and calculated heart rate values (HR);

FIG. 2 illustrates and relates the data points of FIG. 1 the way in which the sympathetic and parasympathetic subsystems of the autonomic nervous system (ANS) of a higher organism i.e. patient, or user mutually affect heart rate variability (HRV) and heart rate (HR) during activities such as reading, sporting, sleeping, etc. The state value (for example flow) can be calculated from it;

FIG. 3 illustrates, for each age, the characteristic average HRV in an average person of that age;

FIG. 4 illustrates a typical beat to beat sequence (RR) of a subject over an A, B, C, D period of time;

FIG. 5 illustrates in flow chart form a process of one format how a real time HRV is calculated from a HRV short value, for which HRV short value is real time calculated per heart beat using 4 RR-periods (A, B, C, D) between 5 (R) pulses, as HRV, and (for example flow) state value determined in accordance with the present invention;

FIG. 6 illustrates in flow chart form a process of one format for simultaneously calculating HR, and HRV (FIG. 5), and determining the state value (for example flow) s determined in accordance with the present invention;

FIG. 7 illustrates in flow chart form a process for calculating real time from heart beat information the (for example flow) state value, (for example flow) state threshold value, and informing or advising the user and possibly logging events in accordance with the present invention;

FIGS. 8-14 illustrate the steps of the process of determining the (for example flow) state value and the (for example flow) state threshold value in accordance with the present invention;

FIGS. 15-16 illustrate the steps of the process of determining the (for example flow) state value and the (for example flow) state threshold value while stimulating the user to enter (FIG. 16) a more happy and relaxed state in accordance with the present invention; and

FIG. 17 illustrates the possible (for example flow) states of a user, which real time per user can be determined by using the (for example flow) state value, low and high (for example flow) state threshold values, HRV value, and HR value in accordance with the present invention;

FIG. 18 illustrates the steps of the process of determining the (for example flow) state value, the related challenge level and skill level in accordance with the present invention;

FIG. 19 illustrates the real time measuring of challenge level and skill level over time, and adaptation of challenge level over time in accordance with the present invention;

FIG. 20 illustrates a scenario of real time measuring HR and HRV over time, and instructing the user to do a different activity (in this case walking) or challenge to optimize flow;

FIG. 21 illustrates the real time display of measuring of the flow over time, including displaying the dynamic determined high and low threshold values;

FIG. 22 illustrates a real time display of measuring of the flow over time, including displaying the dynamic different phases of an activity such as jogging, sprinting, or recovery;

FIG. 23 illustrates a (real time) state indicator by a user interface (over time);

FIG. 24 illustrates other types of (real time or overview over time) state indicators by a user interface;

FIG. 25 illustrates a trigger level for which above the trigger will initialize users behavior change;

FIG. 26 illustrates user state overview indicators over time (per hour, per day) by a user interface; and

### DEFINITIONS AND METHODOLOGY

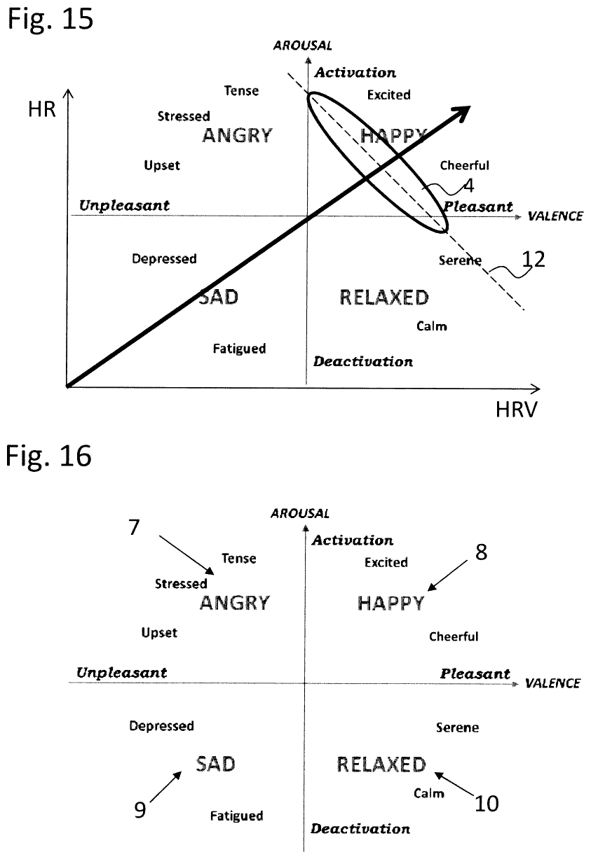

It should be noted that part of the invention is recognizing that a positive state of the user, as is illustrate in FIG. 16 by the right upper part (“HAPPY”) of that graph indicated by the number 8, can be differentiated easily per user from negative state (“SAD”) indicated by the number 9, as is illustrated in FIG. 16 by the left bottom part of that graph, by determining the (for example flow) state value by multiplying the real time HR value with the real time HRV value, as is indicated in FIG. 15. Calculating the (for example flow) state value for the user this way is highly efficient, real time and requires less power and is easy to determine. Any relation, but specific embodiments are here proposed, between HR and HRV can be used to determine a state, another example is for example addition and/or thresholds for both variables HR, and HRV.

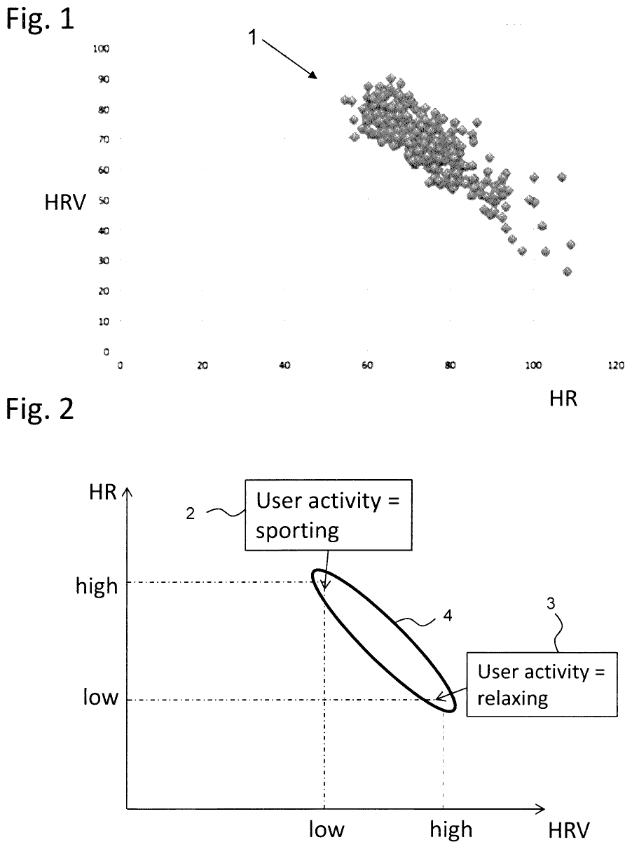

FIG. 1 indicates measured heart beat information data which is used to calculate corresponding HR values and HRV values. Examples of calculated HR and HRV values from heart beat information data are indicated by the number 1. Part of the invention is that a potential relation between Heart Rate (HR) and Heart Rate Variability (HRV) is envisioned. This relation is easier to establish when the user is able to measure and calculate HR and HRV real time as proposed by this invention.

FIG. 2 indicates such a HR and HRV relation, indicated with the number 4, also depends on the activity type of the user while HR, and HRV is real time (or logged and calculated offline) calculated. The elliptical shape illustrates possible positions of calculated and related HR and HRV based upon real time data point measured. As indicated in FIG. 2 for example while the user is sporting indicated with the number 2, as indicated by the arrow pointing within the elliptical shape to sporting data points. For sporting the HR will be higher than normal and the HRV will be lower than normal. Another total different activity as indicated in FIG. 2 with the number 3 is for example when the user is relaxing. For relaxing the HR will be lower than normal and the HRV will be higher than normal, as is indicated by the arrow pointing within the elliptical shape to relaxing data points. This kind of relation can be found. The user can be informed based upon such a relation derived state (for example flow) (value) based upon the calculation of HR and HRV (real time or offline) as proposed by this invention.

The invented (for example flow) state value relation (FIG. 8) can be easily expressed in HR and HRV. The best mode expression of this relationship, to get enough sensing or calculating resolution, is a multiplication of HR and HRV. Alternative ways to determine the (for example) flow state value could be by addition of HR and HRV (which has less resolution) or expressing this relation by defining several (flow; happy, angry, sad, relaxed) states via for example “if HR>a threshold AND if HRV>another threshold, then (for example flow) state value= . . . ”-statements in which such a relation is expressed. In which for example the flow state could be; “worse”, “better” or “good”.

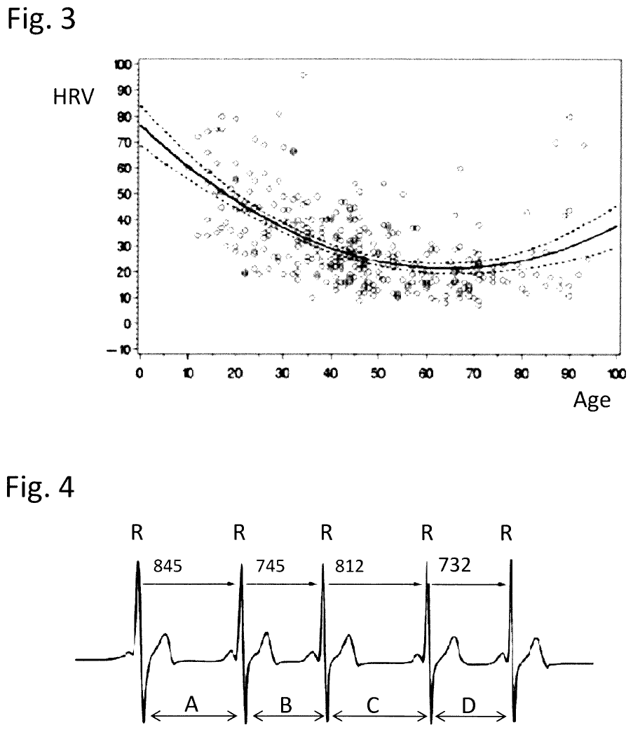

In FIG. 3 is indicated that the relation between HRV and age is age dependent. On average, an average (long term) HRV will decrease over the years for the average person. So the relation between HRV and HR, is on average, age dependent and activity dependent. Probably there will be more user specific dependencies. But average does not work in user feedback systems. It should be real time to be efficient and effective. To investigate such dependent relations it would be very welcome to have a real time calculation method and system for calculating HR and HRV and method to discover such (for example flow) state relations. So the (for example flow) state value relation between HR, HRV and for example activity can be real time determined per user from the heart beat information as proposed by this invention.

State value changes (for example Flow of the user) are in real time and could change very, very fast, as a Dutch saying “Jantje smiles, Jantje cries”, which can be within a fraction of a second. For real time state determination, the system should be really fast and so the method is optimized for real time determination of (for example flow) state value and for it real time calculation of HR, and HRV.

FIG. 4 illustrates a typical example of a beat to beat sequence as an example of heart beat information. Heart beat information can be delivered in an analog signal as indicated here, or in a list of beat data itself, or interval beat data, or any other format. In this example each beat is indicated with a sign R. The period of time between two beats called RR-time varies over time for a healthy user. In this example the RR-times varies for periods A=845 msec., B=745 msec., C=812 msec., and D=732 msec. The average period time between these 5 R-beats is (A+B+C+D)/4=(845+745+812+732)/4=783.5 msec.

One of the embodiments of this invention incorporates a HRV short variation time (the first heart rate variability value), which can be calculated per extra beat. For example by an absolute taken from (period D−average period time (A+B+C+D)/4) is for this example=absolute (732-783.5)=51.5 (delta) msec.

The method to calculate HRV short is an embodiment of this invention. Probably this is one of the fastest methods to calculate HRV. Or just three periods (A, B, C) could be used to calculate an average. Or more heart beat periods could be used to calculate a HRV short in line with the embodiments of the invention presented here. HRV short is calculated by taking some RR-periods (in this example 4 RR-periods) calculating the average period time for these periods and taking the absolute (difference) of a last period(s). The HRV short is calculated per heart beat(s). This period can be expressed in time or in heart beats; for example 2 to 60 heart beats (approximately one minute or less), preferably less than 30 heart beats. Best mode is less than 10 heart beats.

HRV short could be used directly as an HRV value as an embodiment of this invention. It appeared, from best mode point of view, that some averaging over some HRV (short) values (over some time) could result in a slightly better HRV value results for determining a (for example flow) state value. In the following section this calculation is explained more in detail.

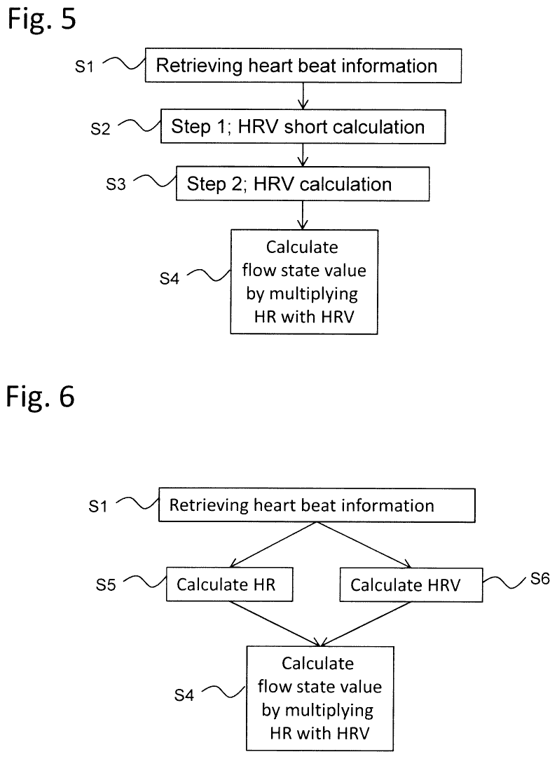

FIG. 5 illustrates this process of calculation of an embodiment of HRV short (FIG. 4) and a derived HRV from HRV short as an embodiment of this invention. From the retrieved heart beat information (step S1) the HRV short calculation can start (step S2);

### Step 1; Example of HRV Short Calculation

HRV short (per third time)=

(Absolute of ((D)−(A+B+C+D)/4)+

Absolute of ((C)−(A+B+C+D)/4)+

Absolute of ((B)−(A+B+C+D)/4)+

Absolute of ((A)−(A+B+C+D)/4)/4)

HRV short value could be used as an HRV value. Another embodiment is to average some HRV short values to optimize the HRV value. This is explained by the following step (step S3).

### Step 2; Example of HRV Long Calculation

HRV long is for example calculated per heart beat by taking the average of the last 10 to 60 subsequent calculated HRV shorts. In this embodiment HRV long is calculated by taking an average of the last calculated sequence of HRV shorts, for example the average of 30 subsequent calculated HRV shorts. The period which is used to determine (for example flow) state value could be predetermined selected in time (approximately one minute or less) or in number of heart beats. The period for calculating HRV long is approximately 1 minute or less, best mode is 40 heart beats or less. The period for calculating HR short is approximately 1 minute or less than 1 minute, best mode less than 10 seconds or less than 10 heart beats. These periods could be predetermined selected and can be represented by a predetermined number of heart beats or in time.

An alternative for calculating HRV short, which is an embodiment of this invention, is using subsequent RR-times from a short period. This short period of time could be a bit longer than the respiratory rate full period of inhale, and exhale period together for example 5 to 15 beats for a average person. For this short period the HRV is calculated alternatively by finding the longest RR-period and finding the shortest RR-period. For example the HRV short value is then calculated as being =(longest RR-period−shortest RR-period)/2. Per beat such a HRV short value can be calculated by means of for example shifting the short period 1 beat further per every new beat taken from the heart beat information. HRV long than can be calculated by averaging the HRV shorts for a longer period of time. For real time applications the short and longer period is shorter than a minute.

Per heart beat (step S4), real time, HRV and HR could be calculated and used to real time determine the (for example flow) state value by for example multiplying HR with HRV.

FIG. 6 illustrates the process of determining the (for example flow) state value (step S4) by an embodiment such as multiplying Heart Rate (step S5) with Heart Rate Variability (FIG. 5; step S2, S3. FIG. 6; step S6) based upon the heart beat information (step S1) with one of the embodiments indicated above for calculating HR and HRV. HR and HRV can be calculated in parallel or sequential. To optimize the HR, and HRV calculation interdependency, the interval period for which both are calculated should be selected the approximately the same period length; for real time applications between 3 and 60 beats for the calculation of both HR (frequency) value, and HRV time value.

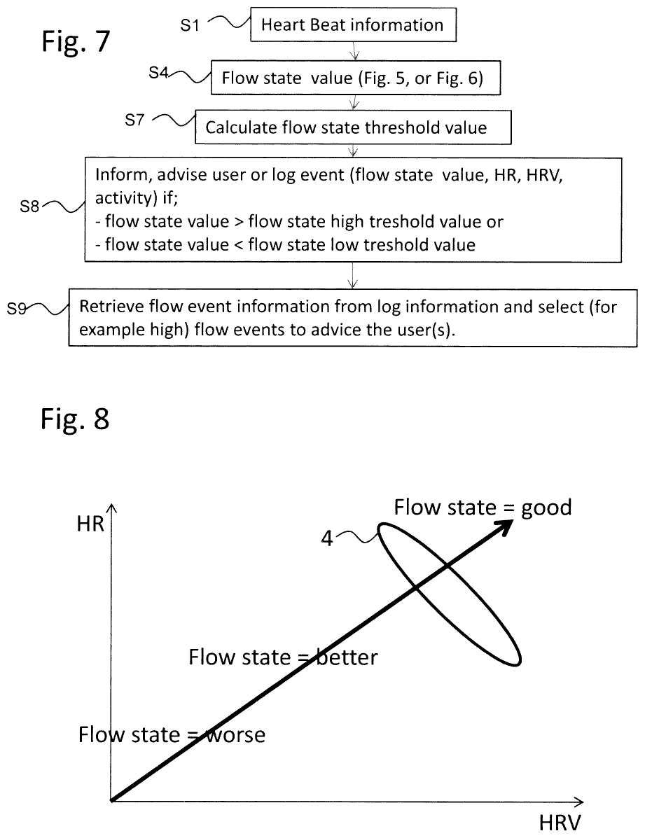

FIG. 7 illustrates the process of determining the real time (for example flow) state value (step S4) from the real time heart beat information (step S1) where after the real time (for example flow) state threshold value (step S7) could be determined by one of the embodiments of this invention. Such a threshold value could be pre-determined or real time determined. Determining these thresholds is further explained by also using illustrates of FIGS. 8-14.

FIG. 8 illustrates a (for example flow) state value, indicated with the number 4 for which value represent “getting better” in the higher HR and higher HRV regions, which can be represented by a higher value or an alternative way such as proposed by this invention. In general a higher value for (for example flow) state value indicates a happier state of the subject. The (for example flow) state value could indicate a worse versus a good state by means of such a (for example flow) state value. For higher or lower (for example flow) states values or states, different states (names) can be defined. To differentiate between those higher and lower (for example flow) state value versus states, thresholds could be defined between such (for example flow) states categories. As an example two such borders or limits of a (for example flow) state (emotion state) class are explained and represented by a low and a high (for example flow) state threshold value in FIG. 10. The low (for example flow) state threshold is indicated with the number 11. The high (for example flow) state threshold is indicated by the number 12. These thresholds separate the flow states ““worse”, “better”, and “good” as indicated in the figure.

Such real time (for example flow) state threshold values are determined based upon the real time (for example flow) state value itself. This is done based upon the idea that each person or user is different in subjects basic (for example flow) state and the adaptability of subjects basic (for example flow) state. So the (for example flow) state threshold initial values must be real time quickly adaptable. And these threshold values should adapt depending on the HR and HRV values progress a user is making during the real time activities in daily life. It should be as real time fast adaptable as life itself is.

The (for example flow) state threshold value is initialized for example by a common average HR=80 beats/minute and average HRV=50 msec., so a common preselected average (for example flow) state value could be selected such as 80*50=4000 beats*msec./minute, but any value selected for example approximately between 2000-8000 could be selected. Based on this an initial predetermined low (for example flow) state threshold value is selected for example 3000, and as initial predetermined high (for example flow) state threshold value is selected for example 6000. It is helpful to make the high (for example flow) state threshold a factor higher than the low (for example flow) state threshold value. For example the high flow state threshold value could be this factor multiplied with the low flow state threshold value. Best mode value for this factor seems to be between 1 and 4, preferable 2. So as an example the high flow state threshold value is 6000.

These initial low and high flow state threshold values will be used to differentiate different (for example flow) states of the user. Now with the determined flow state value the system and method is able for example to determine whether the user is in the flow state “worse”, “better”, or “good”. For example in FIG. 10 the (for example flow) state value indicates that the user is in the state “better”.

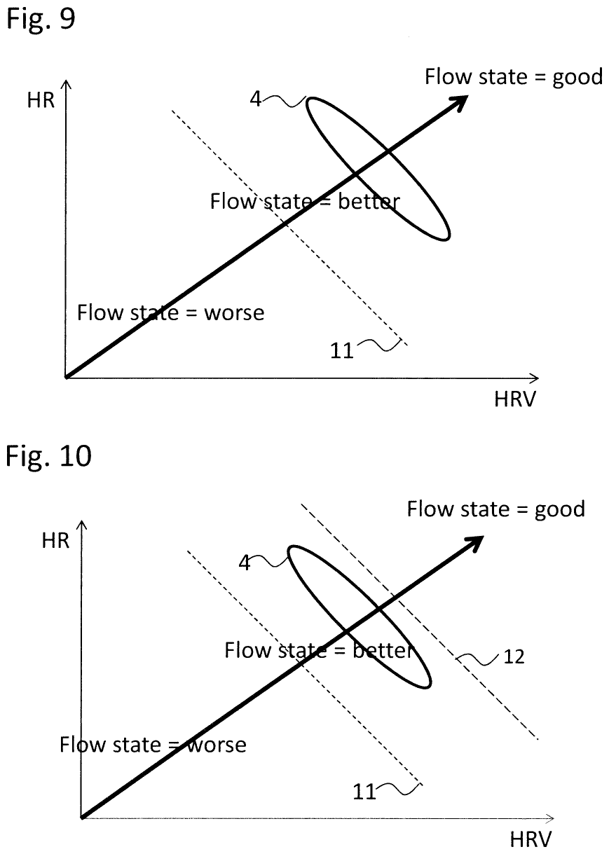

FIG. 9 illustrates to differentiate between the (for example flow) states “worse” and “better” by the flow state low threshold indicated by the small dashed line and the number 11. The oval indicates the real time determining of values of the flow state, indicated by the number 4.

FIG. 10 illustrates additionally differentiation between the (for example flow) states “better” and “good” by the flow state high threshold indicated with the dashed longer lines and number 12. Of course more than two thresholds can be used to detail more than three user flow states.

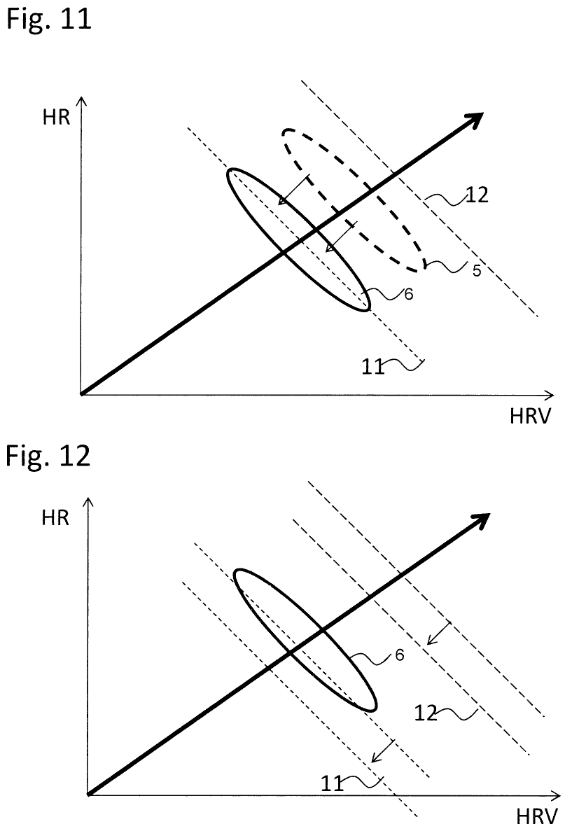

FIG. 11 illustrates while real time determining (for example flow) state values that such a flow state value (dashed oval indicated with the number 5) can real time decrease to a lower value (full line oval indicated with the number 6) indicating a lower value direction by the arrow moving in direction of the flow low threshold value indicated with the number 11. When the flow state value surpassed the low boundary of the flow low threshold value then this is counted for by increasing by one a total of “number of worse heart beats”.

FIG. 12 illustrates that the low and high threshold values are decreased dynamically to a new position 11 (low), and 12 (high) respectively when the (for example flow) state value trespasses the flow low threshold value. The method will be explained later.

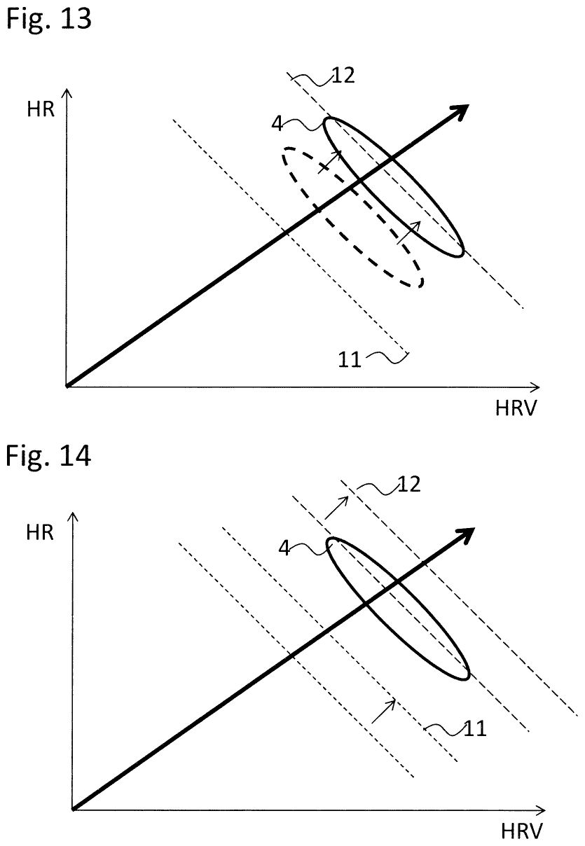

FIG. 13 illustrates while real time determining (for example flow) state value that such a flow state value (dashed oval) can real time increase to a higher value (full line oval indicated with a number 4) indicating a higher value (direction) by the arrow moving in direction of the flow high threshold value indicated with the number 12. When the flow state value surpasses the boundary of the flow high threshold value then this is counted for by increasing by one a total of “number of good heart beats”. In such a case the flow state value moved from the flow state “better” to the flow state “good”.

The ratio between both, being the total of “number of worse heart beats” divided by total of “number of good heart beats”, is a ratio value which is used to determine the new real time flow low and high threshold values as indicated in FIGS. 12 and 14. For example FIG. 13 illustrates that flow state value trespassed the flow state high threshold. In such a case the total of “number of good heart beats” is increased with one. FIG. 12 and FIG. 14 illustrate that when the ration between the totals of good and worse heart beats changes slightly, depending on which threshold (low or high) is surpassed by the flow state value, so that the values of the low and high thresholds increase or decrease.

FIG. 14 illustrates that the low and high threshold values are increased dynamically to a new position 11 (low), and 12 (high) respectively when the (for example flow) state value (4) trespasses the flow high threshold value.

An example of an embodiment for real time adaptation of these thresholds is the following. Flow state low threshold value=3000 multiplied with total of “number of good heart beats”/total of “number of worse heart beats”, and

Flow state high threshold value=2*Flow state low threshold value.

The initial values 3000, and 2 are best mode examples, but different initial values could be selected and having almost the same results. The factor value determines the relative number of threshold trespassing per time. By such a method the flow state thresholds will real time dynamically adapt to the correct flow state threshold position to warn, to advice the user with respect subjects real time flow state and to log important events or activities of the user due to low or high flow state values. It is very appreciated that these thresholds adapt to the specific user state, specific user activities, specific user progress in adapting to the users feedback by such an invention. Based upon big data for specific user types or activity types these thresholds could be preselected.

An advantage by using such a ratio to determine the threshold values is that the total “number of worse heart beats” and total of “number of good heart beats” rapidly increases. The result of this is that the behavior of adaption of these threshold values in the beginning when the total numbers are small is very adaptable, and after some time the total numbers are getting bigger and so the threshold values are starting to get less adaptable. This is the best mode of such a classification system and method for flow state value; thresholds are adaptable in the beginning (best mode several or more heart beats) and getting less adaptable in time (for example more than x heart beats or x time) so that the threshold values are real time user specific and still adaptable in the long run.

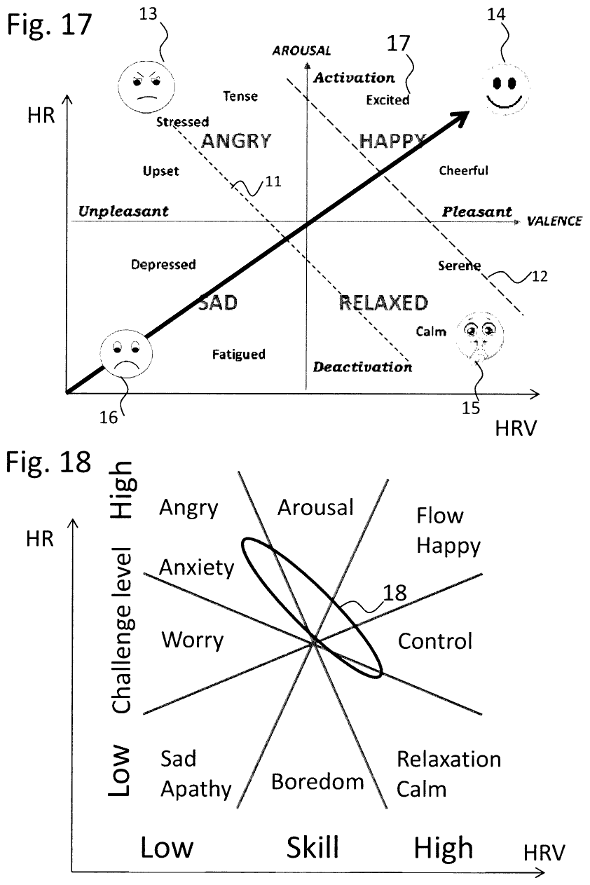

FIG. 7 further illustrates in the case the (for example flow) state value is trespassing a low or high flow state threshold value (step S8) that (an example of embodiment of the invention) it informs the user about it, for example indicating entering the “worse”-state by a visual indication of a “sad smiley” (FIG. 17) with a state indicator “SAD” and a low beep sound. Indicating entering the “good” state by a visual indication a “smiling smiley” (FIG. 17) with a different state indicator “Happy” and a different sound such as high beep sound. Or giving advice (step S8) to the user in the case of trespassing the low threshold about what to do to solve the worse state quickly for example by indicating that the user should “relax” or “change activity”, or take a “deep breath out” by for example words, sounds or pictures, or a video.

Further for example (step S8) the same low or high state event (trespassing of the state value of the state threshold value) could automatically result in logging the users situation in real time. For example logged are the HR, HRV, flow state value, activity, gps, video, photo, sound, etc. to capture the moment. Current (such as wearable) devices have or could have a lot of sensors on board which can be used to register these events automatically by using these sensors to store the event in relation with for example the state value, activity, user state. So that the states could be logged, categorized and sorted. Also states could be derived from the log afterwards in a history overview. In this way the user is able to review progress in states to optimize his learning or just for fun to keep track of high (for example flow) state situations automatically.

One aspect of the invention is logging an event in which a subject is having a too low flow or high flow. Just before such an event for example video, photo, or sound is already continuously captured and is only logged automatically including a certain period of time before such an event is triggered by the flow value while comparing the flow value which trespasses a low or high flow threshold. These events logs eventually can be send real time to the cloud for further services such as alarming, coaching, or big data analysis. A too low flow event could also generate a real time coaching advice such as to warn the user to take it easy and to take a seat during an episode of low physical or mental energy, such as epilepsy risks, heart risks, blood pressure risks or any other physical or mental problems. A coaching test can be built in to check whether the user is able to react to a question in such a situation via the user interface; yes, no, no response.

One aspect of the invention is that such a log could be retrieved real time or offline to sort for example for the high state value and the corresponding items such as activities delivering those high states. A list of high state value activities could be predefined or such a list could be a received, sorted, logged list of (personal or groups) users high state value activities. By this way the user is informed which activities deliver the user high (for example flow) state values. Such information could be very useful especially for user's real time low state situation for which the user can automatically be advised to switch to a specific high state activity which is derived from the log analyses as already explained. In this way the user is automatically advised to switch to a subset of high flow state activities, which are user specific and well known to the user already, which will stimulate the user to real time change and adapt to a higher flow state in a real time matter.

State value and corresponding activity is in a specific real time sequence. Logging of this sequence is very useful (by logging also timing information to the state value, etc.) and part of one of the embodiments of this invention. There are useful or helpful sequences from state value point of view. Finding the best sequence in daily life is not easy for a user. This system can help the user in logging and finding a useful high state sequence. Correlation between “start low state”-activity and switch to the next “go to high flow state”-activity to optimize flow state value is most effective and can be derived when also logging the sequence between these state changes. Big Data techniques can be used to derive useful information from this logged information.

FIG. 7 further illustrates (step S9) an embodiment of the invention that the user is able to review for example the users highest flow states (or lowest flow states) with corresponding logged related items as explained in a sorted list so that feedback is delivered, and possibly specific advice is given or can be derived.

FIG. 7 further illustrates (step S9) in the case for example two user are using such networked method and system and their high flow state value activities are logged and are retrieved automatically and the high flow state activities can be compared to find some mutual high flow state activities. Such a networked method and system can be a help in selecting and proposing some (mutual) high flow state activities and can also indicate which low flow activities should be avoided. Such a system could be of assistance in helping people working in groups, in learning engaging situations, or entertaining situations such as romantic activities. Such a networked method and system can also help people to avoid low flow states such as dangerous high negative stress activities. Such a networked system and method can contribute in the real time dynamics between people. For one or more users getting the users highest flow states in a sorted matter so that feedback is delivered for specific advice is very valuable.

FIG. 16 (taken from; “The circumflex model of affect: An integrative approach to affective neuroscience, cognitive development, and psychopathology”, Dev. Psychopathol., 2005) illustrates an example of different emotion flow states in which the user can be in. On the axes directions emotional states classes are indicated by arousal versus deactivation and on the other axis are indicated by unpleasant versus pleasant. Based on these axes some detailing emotion states can be mentioned. Number 8 indicates a happy state the user can be in with for example sub emotion states excited and cheerful. Number 10 indicates a relaxed state the user can be in with for example sub emotion states serene and calm. Number 9 indicates a sad state the user can be in with for example sub emotion states fatigued and depressed. Number 7 indicates an angry state the user can be in with for example sub emotion states upset, stressed and tense. The invention recognizes that these emotional states can be expressed in combinations of HR and HRV as is indicated in FIG. 15.

FIG. 15 illustrates embodiments of this invention that will help the individual user to increase the flow state value (indicated with the number 4) of the user to a higher state level of happiness by stimulating trespassing their high threshold value (indicated with number 12), and also helps groups of users to do this, to explore the human potential in better and faster real time ways in direction of happy, relaxed, contributing life styles, in which still the control of behavior is the user him/herself.

Privacy in such a system must be kept 100% and under control of the user. With the embodiments of this invention the user can consciously and unconsciously connect more with what the heart wants; better rhythms in the future. The embodiments of the invention will stimulate the user to enter (with the flow state value) more often the happy state by feedback, informing, or advice the user. Flow or state are examples of all kind of possible subject states. Flow is often indicated as an ultimate happiness state in which the user is engaged in an activity. FIG. 15 illustrates specific user flow states (=for example emotion states as indicated) with specific HR and HRV combinations. FIG. 17 illustrates by using the method of this invention that it is possible to real time determine in which emotion state the user is in during the activities.

For example the flow state “happy” (indicated with the number 14) can be determined in case of that the flow state value is approximately in the neighborhood of the high flow threshold value or larger than the high flow threshold value.

For example the flow state “sad” (indicated with the number 16) can be determined in case of that the flow state value is approximately in the neighborhood of the low flow state threshold value or smaller than the low flow state threshold value. To be in the “sad” state, the HR value and HRV value are both in a low zone, so this is also checked before this state “sad” state is determined.

For example the flow state “angry” (indicated with the number 13) can be determined in case of that the flow state value is approximately by a HR in a high zone, while HRV is in a low zone, so this is checked before an “Angry” state is determined.

For example the flow state “relax” (indicated with the number 15) can be determined in case of that the flow state value is approximately by a HR value in the low zone and HRV value is in the high zone, so this is checked before this state “relax” state is determined.

The number of emotion states in the examples above is 4 emotion states (happy, sad, angry, relax). By using additional detailing rules for HRV and HR combinations, additional emotion states (for example indicated with the number 17) can be classified (depressed, fatigue, upset, tense, excited, cheerful, serene, calm) by using the same thresholds as explained. An alternative embodiment is using more than two flow state thresholds or other HR and HRV rules for flow state classification.

The determined emotion state can be indicated visually to the user by a flow state indicator, possibly logged automatically together with the flow state value, HR value, HRV value, and already indicated other parameters, so that the user is able to also retrieve the logged emotion state on a later moment via such a log information.

Another embodiment of this invention is that users can pro-actively limit receiving information (to prevent overload of information) by using an embodiment of this invention which only will present internet-, application-, or news information to the user when the user is in a flow state that is high enough to receive such an information (this is possibly a specific flow state threshold value which could be pre-determined or dynamically established to compare with the flow state value). Sometimes the user is not in a (high enough/good) state to conceive bad state information, and should limit the information to good state information automatically. When selecting a high threshold, the user will (only) get good state information. Information could be predetermined classified with respect to the information flow threshold value based on a test group of people.

FIG. 18 illustrates an example of different emotion (for example flow) states in which the user can be in. Illustrated with an oval (18) a set of real time HR values, and real time HRV values are calculated and illustrated in this figure. The emotions can be measured based upon embodiment of this invention in (relative and/or absolute) values of HR, and HRV. When the users activity challenge level (vertical axe) is high and the users skill level is low (horizontal axe) (for that activity) then the emotion state is anxiety. When the users activity challenge level is high (for that activity) and the users skill level is medium (for that activity) then the emotion state is arousal. When the users activity challenge level is high and the users skill level is high then the emotion state is flow. When the users activity challenge level is medium and the users skill level is high then the emotion state is control. When the users activity challenge level is low and the users skill level is high then the emotion state is relaxation. When the users activity challenge level is low and the users skill level is medium then the emotion state is boredom. When the users activity challenge level is low and the users skill level is low then the emotion state is apathy. When the users activity challenge level is medium and the users skill level is low then the emotion state is worry. The invention recognizes that these users perceived emotional states, challenge levels, and skill levels can be expressed and measured in combinations of calculated real time (relative and/or absolute) HR and HRV values as is indicated in FIG. 18. HR values are indicated on the vertical axes. HRV values are indicated on the horizontal axes. From above embodiment of the invention gamification can be enhanced by controlling the challenge level in the game or activity. It is possible with the embodiment of the invention to be able to fulfill motivation requirements to build the ideal gamification of something one want to learn, by (1) One must be involved in an activity with a clear set of goals and progress. This adds direction and structure to the task. (2) The task at hand must have clear and immediate feedback. This helps the person negotiate any changing demands and allows them to adjust their performance to maintain the flow state. (3) One must have a good balance between the perceived challenges of the task at hand and their own perceived skills. One must have confidence in one's abilities to complete the task at hand.

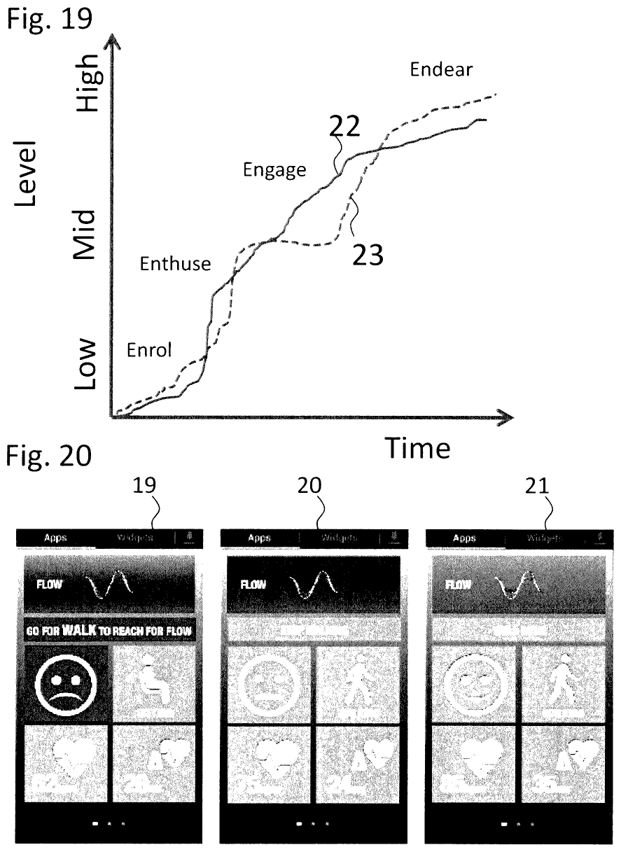

By building a solution (to maximize flow) that avoids for example that users skill level is higher than (for example game or activity) challenge level, which results in an emotion state like apathy or boredom, flow can be optimized. Or by building a solution (to maximize flow) that avoids for example that a challenge level is higher than users skill level, which results in an emotion states like worry, and frustration, flow can be optimized. So with the invention's embodiment, for example during a game or an activity, the game or activity can be optimized on challenge level and skill level by real time measuring users heart information and calculating users real time HR and HRV. By which real time the user's state and skill level is determined and by which controlling in dependency the challenge level events or challenge level sequence which is imposing upon the user to optimize flow. An example for gamification embodiment of this invention is given by for example playing pacman. When the skill level of the user is high (represented by a high level of HRV value) (and relative low HR value) by an embodiment of this invention the number of ghosts is increased, and as a result some extra challenge level is added. The users or players challenged level due to this can be measured by an embodiment of this invention and probably will increased as can be seen by a higher HR value of the user and a lowering of HRV value of the user. When this challenge level increase has a high impact (which is measured) then the embodiment of the invention method can slow down the velocity of the pacman so that the challenge level is decreased a bit (which is measured). The envisioned result of this invention is that the motivation level of the player is kept high (and flow level) and the abilities of the user is increased faster, resulting in a higher skill level, which is indicated by a lower HR value and higher HRV value (which is measured) for this activity in a shorter period so that thereafter via an embodiment of this invention the speed of the eating of the pacman can be increased to sustain a higher flow level during the activity. From a point of abstract thinking the following philosophy is supported by an embodiment of the invention; heart rate frequency can be seen as a quantity level of energy transport and heart rate variability can be seen as a quality level of energy transport (pumped by the heart) through body and mind. Based upon the measure method represented in FIG. 18 skill and challenge level scenarios can be optimized. For calibration and initialization purpose the maximal skill level, and/or minimum challenge level of the user can for example be found during sleep, deep relaxation, meditation or via Big Data analysis. The highest heart rate variability value of a user is often found for example during sleep for which most people are highly skilled. For calibration and initialization purpose the minimal skill level, and/or maximal challenge level of the user can be found for example during new learning of new activities or via Big Data analysis. As an example FIG. 19 indicates such a scenario. On the horizontal line the time of the scenario is depicted. And on the vertical axe are both skill level (skill values indicated by 23) and challenge level (challenge values indicated by 22) indicated with respect to low, mid, or high (skill, challenge) level. The measured real time users skill, and challenge values based upon real time measurements of HR and HRV, are illustrated for such a scenario, for which the challenge level values (full line 22) is updated per heart beat with respect to the real time users skill level values (dotted line 23) so that the user can go through time via for example the indicated emotion states such as enroll, enthuse, engage, and endear, while keeping a high flow state.

In FIG. 19 is illustrated the different phases of engagement during a game or activity for the skill level versus challenge level engagement also called here “flow-DNA”. Both skill level and challenge level are indicated on the vertical axe of FIG. 19. An embodiment of the invention is to optimize skill level and challenge level in such a way that the challenge level (hear rate frequency level) and skill level (heart rate variability level) match each other to keep the user engaged in the real time activity. Herewith an example is given for such an activity such as playing a sport like football. During starting the activity the HR & HRV (=for example 60 beats/minute*60 msec) will result in the phase; “enroll” (just start the activity), then after a while the phase enthusiasm must be gained; a better HR/HRV ratio is required (for example HR*HRV=100 beats per minute*35 mseconds). Thereafter the phase engagement must be stimulated in which flow is going to an optimal position for maintaining that activity type on the long run during the game or activity (for example flow=HR*HRV=110 beats per minute*25 mseconds; for example for football). And finally for the peak states during the play endear phase could be stimulated (for example flow=HR*HRV=150 beats per minute*20 mseconds), during a sprint for a winning goal. By the ability to measure and train for such states this embodiment of this invention creates real value within the activity; something unique is happening. Gold medal winners are in this state of performance. Capabilities are optimized; stimulating peak performance via challenge level and skill level matching over time as indicated in FIG. 18. in relation with FIG. 19.

An embodiment of this invention calculates a flow that will fluctuate per beat over time. To be able to optimize for flow only max flow is generated when max flow is required for a high challenge level. Of course maximal flow value and/or HRV value recovery in flow must be optimized for during recovery also as part of the game or activity. Especially after an overreaching activity (the challenge level was too high with respect to skill level) in which the HR frequency is increased very fast (during a sprint) and the HRV cannot compensate for the loss of flow. Then there should be a flow recovery time to be able to continue the game or activity and to recover fast to have no negative effects on the long run on health. How we do this, needs some additional explanation. During exercise or training the training should also incorporate maximal recovery during the exercise to recover from the activity. For this the ratio average flow value over time is an important factor which should be above a certain threshold to keep the body & mind in a healthy state. For example the average flow over time should be higher than 30. To further elaborate, the recovery over time, FIG. 22 illustrates a scenario for example a football player during a match. In such a game there are phases in which the player must sprint for the ball and moments in which the player can recover from the sprints. In FIG. 22 the sprint periods are indicated by numbers 29, 31 and 33. The flow recover periods are indicated by numbers 30, 32, and 34. On the vertical axe of FIG. 22 the real time flow is indicated representing the real time calculation of heart rate frequency multiplied with heart rate variability. A minimum level of flow is indicated with line 27 which is for example is a flow of 30 (Flow value=HR*HRV/25). A high flow level is indicated by line 28 which is for example a flow level of 100. During the activity an embodiment of the invention compares the real time flow level with some thresholds such as a minimum required flow level, a minimal flow level recovery change per time (so the flow must recover fast enough in period 30, 32 or 34), and/or the maximal recovery total time. For example in period 34 the flow level recovery change per time is not high enough and therefore the player is not able to gain flow above a minimum flow level. Also the duration of the recovery period in total time is too long. The player or coach get instant feedback in such a situation to give this player more recovery time to optimize restoration of energy and health. The flow level during the activity should be higher than a minimum threshold. The flow (recovery) change per time should be higher than a minimum threshold during recovery. The total flow recovery time should be smaller than a maximum total flow recovery time threshold. When one of these parameters is inadequate, potentially overtraining might occur. In such a case, to optimize a minimum level of fitness, immediately the user of this embodiment is advised to restrict workload frequency, intensity, or duration of training or activity. To improve flow, in real time, advice via suggestions can be given by the embodiment of this invention for example when the average flow over time is less than a certain threshold. An embodiment of this invention is to determine specific advice based upon the real time heart rate frequency value and real time hear rate variability value to optimize peak flow (for example for interval sports sometimes over stretching will occurs to win the game), (average) flow over time (for example for duration sports to optimize for an energy conservation on the long run), or flow recovery time (for example after sporting or during interval sports to recover during the game itself). So part of the embodiment of this invention is to optimize skill level of the user per beat which is dependent of HRV level and challenge level for the user due to external factors which is dependent on HR level. During a game or activity a specific performance goal is planned for for example running a distance value over a time value with a certain resistance value (for example percentage climb or sprint). The HRV and HR can be scheduled to be optimized during such a performance goal plan. Advice in relation with the performance goal plan can be real time generated based upon the HR and HRV values. Afterwards the achievement (distance, time, and resistance) and logged HR and HRV values can be compared with the performance goal plan.

Another type of examples is advice when the flow is substantially too low. For example “a cold”-dip suggestion for an average flow over time below 20 could be given. For example taking a cold shower to enhance life energy could be part of this; it is part of an embodiment of this invention that flow can be measured real time also during cold showering and as a result of “the cold” it appears that at least a higher flow is sustained for 30 minutes after taking a cold shower for example. Care must be taken before advising taking a cold shower whether the health of the user is high enough to be able to advice this; so advices are given based upon for example a specific thresholds with respect to HR/HRV ratio over a longer period of time and/or depending on the flow level. This kind of advice will probably work for healthy people having enough flow-capacity in body in mind due to training physics/emotional balance. Big data analyses will make clear which specific advice will result in which specific potential enhancement in HR/HRV and/or flow. For example taking a-“cold”-dip as part of the daily activity and/or as part of training will also train “will power=(brain power)” to focus on “only” positive outcome results and keeping flexible against meanwhile resistance (=against stress reaction) on the body and mind. It will train the mind and/or body to keep the HRV high in high resistance environments (for example “cold” training). As a result the high HRV state and so high flow state will enhance the use of the brain mirror neurons and keep the mirror neurons in the “on”-state, which will finally result in that users will make better life decisions.

Another embodiment of this invention is to derive the blood pressure in a real-time easy way. In general the following blood pressure formula yields;

Mean Arterial Pressure=Heart Rate frequency*Stroke Volume*Total Peripheral Resistance.

Mean Arterial Pressure (MAP) represents a specific blood pressure calculation method via this formula. Part of the insight of this invention is the understanding that a real time HRV calculation which is an embodiment of this invention is an indirect measurement of real time heart Stroke Volume variation. Part of the insight of this invention is the understanding that a real time Heart Rate Frequency represent the mid frequency of the heart, a real time Heart Rate Variability represent the full width frequency range of the stroke volume variation of the heart, and the Total Peripheral Resistance represent the low frequency stress-variation (or resistance) of the body & mind & heart-system. Based on these insights it is possible to derive an embodiment to determine the blood pressure on an easy way. The real-time relation between real-time HR frequency and real-time HRV indicates the level of real-time Total Peripheral Resistance. So based upon the both real-time variables HR and HRV the real time TPR (Total Peripheral Resistance) can be derived.

MAP=HR*(SVo+SW)*TPR=

MAP=HR*(SVo+HRV)*TPR=(HR*SVo+HR*HRV)*TPR=

MAP=(HR*SVo+Flow)*TPR, for which Flow=HR*HRV.

A high flow indicates a high adaptability for possible external or internal change requests necessary and still able to stabilize the blood pressure sufficient. A too low flow indicates a low adaptability for possible external or internal change requests. For low flow values TPR is not able to compensate for (high or mid frequency) pressure changes due to HR changes and so there is a higher risk the MAP will fluctuate more, especially in high external adaptability stimulus situations. So for low flow levels a higher heart risks appears for high adaptable requests such high energy tasks as moving easily or thinking clearly; people should slow down when having a low flow; and enhance flow fast by specific personalized increase flow advices.

Heart Rate frequency value is the Medium Frequency value (MF value) of the heart beat information that passed the MF-filter. Heart Rate Variability value contain all frequency of the

Stroke volume variations. Heart Rate Variability value is the High Frequency value (HF value) of the heart beat information that passed the HF-filter. TPR value is the Low Frequency of the blood pressure variations. TPR is the Low Frequency value (LF value) of the heart information that passes the LF-filter.

Another embodiment of this invention is helping a user to be able to perform within their abilities the best way. Proposed is to use the (for example flow) state value and a pre-determined or adaptable sequence of activities within a specific training, professional, medical, leisure or any field for example biking, swimming, fitness, work process, leisure, dating, treatment in which the user acts, and presenting to the user when to go to the next activity within the best (or next) flow state sequence for such a field of activity. As an example, FIG. 20 indicates such a scenario. In the left part of FIG. 20 (indicated by 19) a user is in a sad emotional state and (i.e. sitting) inactive physical state; both states derived from measuring real time heart rate information and real time calculating based upon HRV and HR values. An example of when the user is having a heart rate frequency of approximately (which could be user dependent thresholds) 60 beats/minute<HR<75 beat/minute, it is very likely that the user is sitting. And the emotional state “SAD” is very likely when having a flow during sitting of HR*HRV/25<30. Depending on the ratio HR versus HRV the emotion state can be made more specific in direction of apathy or fear. Based upon these states advice is given to the user to go for example for a walk to reach for a higher level of flow (indicated by colors; higher level of flow is more green for example). In the middle part of FIG. 20 is illustrated that the user has responded to the suggested advice the system had given and that the user due to that is in a more neutral emotional state (by the neutral face indicator) and in a more active physical state (indicated by the walk indicator) both states are measured and determining by measuring based upon the heart information and calculating real time HR value and HRV value in combination. In this case the heart rate frequency is approximately (could be user dependent thresholds) 74<HR<110 beats per minute. The real time measure HRV is increased from 20 mseconds (19 right bottom indicator of FIG. 20) to 24 mseconds (20 right bottom indicator of FIG. 20). The emotion indicator is going to neutral to indicate a more positive state from “SAD” to “neutral”. The physical indicator changed from sitting state to the walking state. In the right part (21) of FIG. 20 is illustrated that the user is still walking with the physical indicator, and the user is given feedback that users flow level is the good state by means of the indicator well done, the green colour of the flow indicator and the emotional state indicator “happy”. These states are based upon the real time HR, HRV combinations. By this intervention scenario for this user the flow state changed from low (19; flow value=HR*HRV/25=62*20/25=50), middle (20; flow value=75*24/25=72) to a higher state of flow (21; 85*35/25=119). The factor 25 is used to scale the flow value. The emotional state indicator could be a relative indicator to indicate the flow change over time. Also here in FIG. 20 for this scenario the real time determination of emotional state (indicated by the smile symbol) and physical state of the user (indicated by the walking symbol) is based upon real time heart information and real time calculation of HR value (indicated on the left bottom of all three parts) and HRV (indicated on the right bottom of all three parts) is indicated. The real time HRV value for this scenario changed from 20, via 24, to 34 milliseconds. The HR value for this scenario changed from 62 beats per minute, via 75, to 85 beats per minute. Another example for state calculation is given by the following thresholds and examples for such thresholds could be for example for a sporting application or a more general stress reduction application. Herewith for example thresholds for warning are explained in more details (in below examples flow=HR short*HRV long);
If the HR short<96 beats/minute assumed is that the mind&body are in a not sporting state
If the HR short>95 beats/minute assumed is that the mind&body is in a sporting state

Physical states examples via thresholds (HR short is for example per 7 heart beats);
If HR short<96 AND flow<2000 then physical state=“stress; relax mind”
If HR short<96 AND flow>3000 then physical state=“Relaxed”
If HR short>95 AND flow<1500 then physical state=“stress; relax body”
If HR short>95 AND flow>2000 then physical state=“Relaxed”
Otherwise physical state=“neutral”

Emotional (relative) states examples via thresholds;
If HRV short>(HRV long+15) then emotional state=“More pleasant”
If HRV short<(HRV long−25) then emotional state=“A bit less pleasant”
If HR short>(HR long+4) then emotional state=“More active”
If HR short<(HR long−4) then emotional state=“Less active”
if HRV short>(HRV long+15) AND HR short>(HR long+4) then emotional state=“More happy”
If HRV short>(HRV long+15) AND HR short<(HR long−4) then emotional state=“More relaxed”
If HRV short<(HRV long−25) AND HR short>(HR long+4) then emotional state=“A bit less Happy”
If HRV short<(HRV long−25) AND HR short<(HR long−4) then emotional state=“A bit less Relaxed”
Otherwise emotional state=“Neutral”

Advice based upon derived states (HR long is for example per 30 heart beats);
If HR long>60 AND flow>3000 then Advice=“Sport/work/hobby ready”
If HR long<96 AND flow<3001 then Advice=“Relax & rest & increase flow”
If HR long<96 AND flow<2500 then Advice=“Deep breath, hold, tighten muscles 10 seconds, then relax”
If HR long<96 AND flow<2000 then Advice=“Stretch muscles, then relax”
If HR long<96 AND flow<1500 then Advice=“Cold shower”
If HR long<96 AND flow<500 then Advice=“Check your status”
If HR long>95 AND flow>2000 then Advice=“In sport zone”
If HR long>95 AND flow<2000 then Advice=“Breath via nose”
If HR long>95 AND flow<1500 then Advice=“Rest”
If HR long>95 AND flow<1000 then Advice=“Relax and slow down”
Otherwise advice=“Neutral”

Part of the invention is the insight that heart rate variability is a build in main task of the heart to pump blood in such a way that it also compensates for (potential) blood pressure changes. The heart compensates for (potential) blood pressure changes via cardiac Stroke Volume adaptation (what is measured as HRV). So that in the end blood pressure is kept healthy within a healthy person. For an unhealthy person this function is jeopardized in such a way that is measurable by for example a change in a combination per beat of HRV and HR. Another example is for blood pressure state calculation is given by the following thresholds. For example such blood pressure thresholds could be useful for example for medical home care, so that the user can be monitored at home. Herewith for example the thresholds (predefined or user adaptable) for warning or logging are explained in more details. Blood pressure warning states examples via thresholds (while for example sitting);
If HR long>84 AND flow<500 then blood pressure potentially not ok.
If HRV long<5 AND flow<450 then blood pressure state=“high”
If HRV long<5 AND 500<flow<800 then blood pressure state=“low”

Advice based upon derived states;
If flow<800 then Advice=“check your status i.e. blood pressure”

An embodiment of the invention is optimized for detecting stress problems. The following algorithm is constructed based on the vision that there is a minimum healthy flow average per short time period. For this embodiment example the minimum healthy flow average per short time period is taken 1% of the pulse-pulse-time of the heart beat information. What the exact minimum percentage is is still under investigation and could be personal depending on the type of mind/body state.

An embodiment of the invention is optimized for detecting arrhythmia problems. The following algorithm is constructed based on the vision that there is a maximal healthy flow average per short time period. For this embodiment example the maximal healthy flow average per short time period is taken 10% of the pulse-pulse-time of the heart beat information. What the exact maximum percentage is is still under investigation and could be personal depending on the type of mind/body state. Two versions of this algorithm are stated here. Version one; for all successive 30 calculated flow per beat then the individual flow per beat should be below a maximal Flow per beat=Heat Rate frequency value*(multiplied to) maximal % of Heart Rate Variability value=HR*10% of (60/HR). The to be determined maximal % of Heart Rate Variability value could be between 5% and 40%, likely it will be between 10% and 25%. The number of successive calculated flow per beat for this algorithm (and the following algorithm) could be between 5 and 60 flow beats).

An alternative embodiment of the invention is version two; that for all successive 30 flow calculations per beat the total of 30 successive flow calculated values should be lower than a maximum total summation threshold or multiplication threshold.

Another embodiment of the invention can determine an user health issue event called ventricular tachycardias which is based on a higher than normal HR frequency in an inactive state (this can be found by combining state detection with an accelerator). The heart is able to generate cardiac output, but is not functioning optimally, and there is a chance that the heart will go to an atrial fibrillation state.

Another embodiment of the invention can determine a user health issue event called atrial fibrillation which is based on real time HR and HRV combinations, which can be determined by a real time too high HRV in combination with a HR frequency variation that is bigger than for example 1 standard division of the normal HR frequency variation.

Another embodiment of the invention can determine a user health issue event called ventricular fibrillation which is based on real time high HR with variable high HRV. In general stated a too low flow or too high state of flow value indicates an unhealthy state of the user and when the user is in a too low flow value or too high flow value measured by an embodiment of this invention then the system will advice to check the status of the user to be able to intervene and to get the user again in a healthy stable situation. Big data analysis could be used to find a cause relation and find a good solution to advice the user or caretakers what to do to get the user again in a healthy stable situation. A specific high flow state indicates a high healthy situation for the user. Big data can be used to understand cause relation of this high healthy situation for the user and store such analyses results to be able to advice the user in the future to be able to generate again such a high healthy situation for the user.

A further other embodiment of this invention is for example helping a user to be able to perform the best way within their own abilities by adding resistance or challenge level to an activity, such as making the sporting challenge for a healthy trained user higher, by selecting a higher resistance or challenge level (for example; kg, length in time of high impact training, length of distance, etc.) automatically. Or for example selecting a lower resistance level or lower challenge level for a not trained less skilled user. Resistance, or challenge level is selected based upon the (for example flow) state value, by determining;
for example the flow state sequence the user is in, and
selecting a training resistance based upon the flow state value for which the user best can train, and for example presenting to the user what to do and when to go to the next activity within the flow state sequence.

Additionally, the present invention is applicable during pregnancy measuring the health of mother and baby. During pregnancy within mother's heart rate information also the babies heart rate information could be derived within the babies normal approximately 120 to 195 beats per minute area (approximately after week 6). From the heart rate information both mothers and babies state can be determined. And for example mutual, external, or internal factors on both states can be investigated via Big Data cause analysis.

Additionally, the present invention is applicable during life threatening situations measuring the health of the patient, or warier in combat. For example such as a comma patient after a car accident, or soldier in war zone. In such a situation real time user flow state determination and/or advice could be life saving. For example to find treatment for a comma patient which results in a positive flow change indication for the comma patient for a specific treatment gives feedback to continue this treatment. Additionally for sport coaching advice and sporting monitoring of players of a team sport the health state can be checked during training or the high performance game to secure a healthy situation. During training situations for example the stimuli (to optimize for challenge versus skill and motivation) can be triggered in sequence of the flow indicators such as selecting the right music type and music rhythm during running; the music bass frequency (ie drum) is selected such that it corresponds to the HR frequency and the higher frequency parts of the music is selected based upon the HRV value or flow value. Additionally for health monitoring for animals in economic situation as a cattle can be use to check the flow states of individual cows of a big stock of cattle automatically online on distance. Additionally, the present invention is applicable to impulse control, providing training to help overcome eating disorders, anxiety, anger, and/or other addictions. To stimulate real time well-being in personal activities, contributing real time to the wellbeing of the individual person. The present invention is beneficial in applying or coping with sport resistance management, stress management, and emotional self-management. The warning or feedback threshold values for such a embodiment of the invention method, for example to stimulate a healthy lifestyle, are determined with Big Data analysis for example Watson technology for health care or any other care or advise. By this way unhealthy user behavior can be detected based upon thresholds determined based upon relating best practices of certain activities in relation with corresponding logged real time HR and HRV (Big Data) database analysis which results in best practices thresholds.

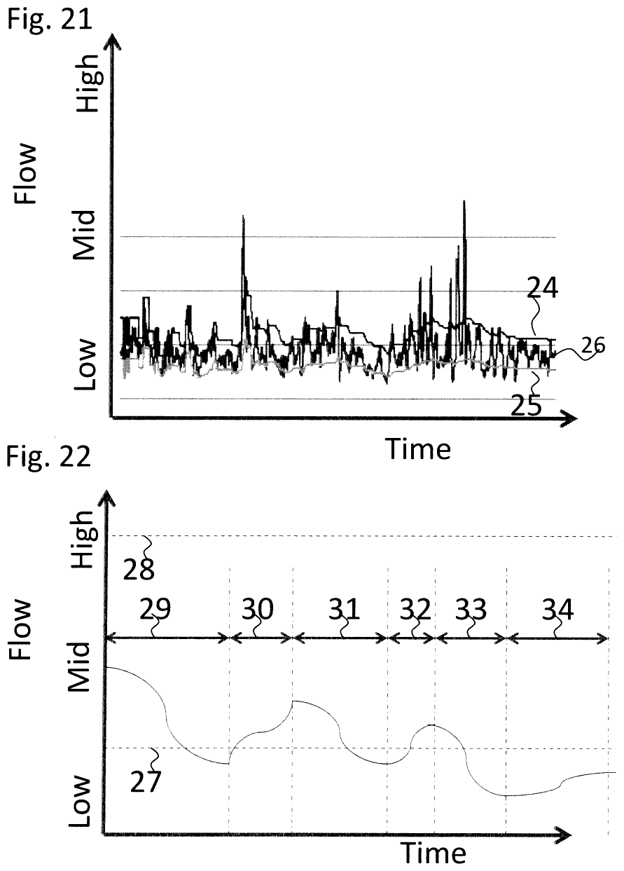

A method that automatically stimulates an adaptable and proactive life style, by advising the user real time for example propose a different user specific activities. An application, method, network, and system that will contribute in striving for individual specific fulfillment of the individual capabilities of a user by feedback and advice. For example making good decisions can be done more easily for higher flow levels (HR, HRV combinations) in context of the real time measurement method as disclosed by an embodiment of this invention. For example the embodiment is generating real time advice and indicates when and only then to make decisions when a threshold flow level is surpassed or certain HR and HRV combinations are reached. By this method the ratio between user's activity (during the activity) capabilities and incompetent can be real time measured. By measuring this flow state value, real time is (indirectly via the heart) measured the amount of (mind and body flow characteristics); clearness of target activity to execute, the level of focus and concentration for the activity, the balance between competence and challenge for the activity, and the level of intrinsic reward. By giving positive directive real time feedback (as an embodiment of the invention) the previous described flow characteristics (real time HR and HRV combinations) can be real time optimized. In FIG. 21 is indicated for an embodiment of this invention the measured and calculated flow (vertical axes; low, mid and high level) values (indicated with 26) over time (horizontal axes). Also the adaptive high threshold (indicated with 24) and low threshold (indicated with 25) are dynamically measured based upon the real time flow value and both are displayed in this figure. Not indicated in a figure are illustrations that are also based upon an embodiment of this invention such as status (change) over time for example to monitor the (real time) physical status or emotional state of the user. For example for sporting a flow (=HR*HRV/25) higher than approximately 15 seems to be advisable (the exact value could be individual determined or via big data analysis determined) and a value lower than approximately 10 should be avoided. Feedback to the user of flow value warnings related to such thresholds and advices what to do in such case is part of an embodiment of this invention.

A method that automatically stimulates an adaptable and proactive life style by increasing flow that is secured in a partly decentralized fashion as a standalone decentralized end-user system such as a smart sport watch or app on a smart phone and partly centralized and secured as a (on request) big data system, which is envisioned and part of this invention and an embodiment of this invention. Flow is envisioned as life currency so that an individual life currency in total can be described as HR frequency value*HRV value*number of heartbeats in life. So the exchange of life currency can be seen as a potential trade for tasks needing specific skill and challenging tasks. The task level requirement can be described as =skill level*challenge level required. It is envisioned that challenge level has a relation with HR frequency level, and skill level has a relation with HRV level. In this way users real time flow level (=HR*HRV=challenge level*skill level) indicates the real time challenge level and skill level one is capable of for the real time task the user is doing at that moment. This way employment vacancy tasks or requirements could be tested by real time measuring HR and HRV levels during executing the tasks. To be able to implement such a system and communication architecture is an embodiment of this invention. To be able to centralize the big data system with respect to user flow on a large scale incorporating millions of end users in parallel using such a system, just two real time parameters per user per timeframe (such as heart beat) could be send into the cloud; heart rate frequency value and heart rate variability value for example per heart beat or per timeframe. Alternatively every hear rate pulse pulse time per beat is send to the cloud, and on the cloud side the heart rate variability, heart rate frequency, and flow could be derived based upon the heart rate pulse pulse time per beat. Technology for sending heart, heart rate frequency, heart rate variability, flow, state information, heart pulse pulse time into the cloud are for example client-server network, or peer-to-peer networks, or chain network. The human private flow state information should be send private and secure. For this blockchain technology seems to be a valid choice.

An embodiment having mutual dependent or independent, an decentralized and centralized part, for such embodiment there is a need for a communication interface which is secure, and easy in maintenance. For this the following embodiment is developed. The core advantages of the block chain architecture include the following:

The ability for a large number of nodes to converge on a single consensus of the most up-to-date version of a large data set such as a ledger, even when the nodes are run anonymously, have poor connectivity with one another, and have operators who may be dishonest or malicious.

The ability for any node that is well-connected to other nodes to determine, with a reasonable level of certainty, whether a transaction does or does not exist in the confirmed data set.

The ability for any node that creates a transaction to, after a certain period of confirmation time, determine with a reasonable level of certainty whether the transaction is valid, able to take place, and become final (i.e. that there were no conflicting transactions confirmed into the block chain elsewhere that would make the transaction invalid, such as the same currency units “double-spent” somewhere else). A prohibitively high cost to attempt to rewrite or alter any transaction history. An automated form of resolution that ensures that conflicting transactions (such as two or more attempts to spend the same balance in different places) never become part of the confirmed data set. A block chain implementation consists of two kinds of records: transactions and blocks. Transactions are the actual data to be stored in the block chain, and blocks are records that confirm when and in what sequence certain transactions became journaled as a part of the block chain database. Transactions are created by participants using the system in the normal course of business (in the case of cryptocurrencies, a transaction is created anytime someone sends cryptocurrency to another), and blocks are created by users known as “miners” who use specialized software or equipment designed specifically to create blocks.

Users of the system create transactions which are loosely passed around from node to node on a best-effort basis. The definition of what constitutes a valid transaction is based on the system implementing the block chain. In most cryptocurrency applications, a valid transaction is one that is properly digitally signed, spends currency units from a known valid wallet, and meets various other requirements such as including a sufficient miner “fee” and/or a certain time elapsed since the currency units were previously involved in a transaction.

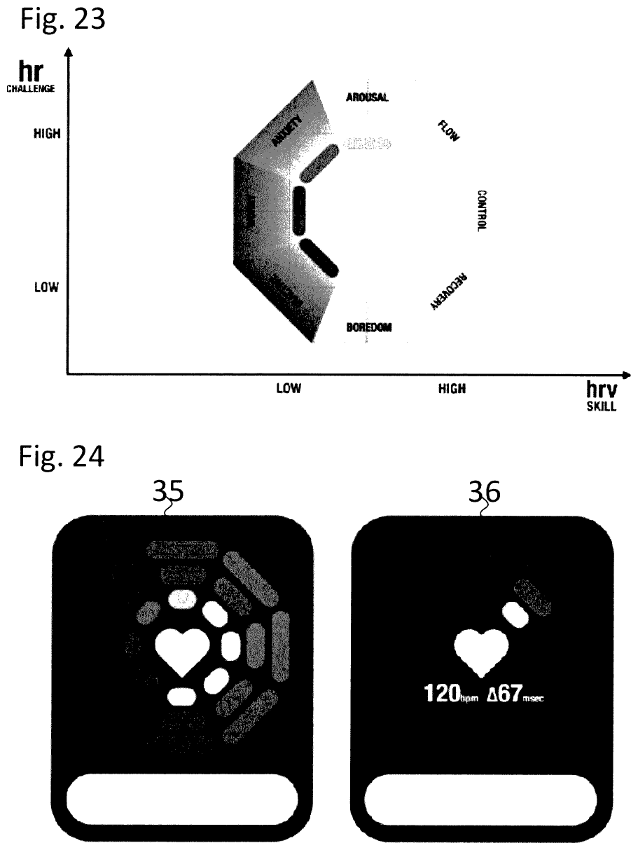

An example of an embodiment for the local or the (cloud) remote user interface will follow. This user interface should be clear, fast and convenient for, for example, the user, coach or doctor. For example it is convenient to see the state per time unit for example per minute, per quarter of an hour, 6 hours, or per hour. Then the indicator could be the percentage of time or an amount of time for which the state value is above a maximum log threshold value and/or under a minimum log threshold value. An example for the maximum log threshold level is a 20% higher level than the high threshold as explained earlier. An example of the minimum log threshold is a 15% lower level than the low threshold as explained earlier. The exact % could be predefined or for example adaptable per user. To remove some very fast changes from the general state view a minimum threshold in time could be incorporated so that too fast changes are not incorporated in the logging or viewing locally or remotely. An example how to do this is for example by only including the state value in this calculation in case of a minimum number of consecutive state values that are above this maximum log threshold and/or below this minimum log threshold. An example of a very convenient way of representation of the users state is the following by determining an ordered list of periods (per minute, quarter, hour, 6 hours or day) based upon the percentage of time or the amount of time for which the state value is above the maximum log threshold value and/or the state value is under the minimum log threshold value or within that state. The ordered list can be represented as a top 3, top 10 or any other way of representation so that the list is ordered in a way the user, coach or big data can find and analyze the important states of the user during a longer period, and understand in a fast way the important smaller periods within the longer period for which a high or low state is logged. Herewith the state can also be replaced by for example the physical state, emotional state, or absolute emotional state. This information (for example state or ordered list) can be sent from the local measuring device such as smart watch or smart phone into the cloud for example logging, displaying, or big data analyses. A local or remote User Interface concept is represented in FIG. 23. FIG. 23 represents an embodiment of this invention. In FIG. 23 on the vertical axe the heart rate frequency (which also relates with challenge level) and a personal (real time adaptable) low and high heart rate frequency value is depicted. On the horizontal axe the heart rate variability (which also relates with skill level) and a personal (real time adaptable) low and high heart rate variability value is depicted. By means of the colour optical densities or grey tones, the level of different emotion types of the user can be represented in such a user interface concept. An alternative representation of this user interface concept is indicated in FIG. 24 by means of the colours and angles in which the colours are represented. The optical density of the colour (or emotion) indicator or the amount in which the indicator is out of the middle of the heart could indicate the amount of emotion (indicated by 35). The real time indicator of the current state (physical, emotional, or absolute emotional state) could be represented by the colour and angle at that moment (indicated by 36). This is an example for a real time indicator of the physical and emotional state of the user. Another embodiment of this invention is indicating the physical and emotional state of the user over time such as indicated in FIG. 26, which could be represented on the local user interface or an a user interface in the cloud. In this embodiment the user state is represented over for example 6 hours, or a day time period or week time period. As such the real time state of the user is summarized per quarter, per hour, per day, per 6 hours or per week. For example on Monday as is indicated with the number 40 the user started with sleeping from approximately 22 hours (Sunday evening) to 7 hours (Monday morning). This sleep state can be derived from the real time beat to beat information by recognizing the sleep state via HR<70 beats per minute and HRV>100 milliseconds (or low sleep state could be represented by HR<70 and flow>200, and deep sleep state by flow>300), or which could also be identified as recovery heart beats for the whole day. The number 41 indicates the active state after sleeping starting from 7 hours to 11:30 hours. This state can be recognized from real time beat to beat information for example by a state like HR>69 beats per minute and HRV>50 milliseconds. The number 42 indicates an eating state from 11:30 hours to 12:30 hours. This state can be recognized from real time beat to beat information for example by a state like HR<80 AND HRV is slowly declining to a lower state due to fullness. The number 43 indicates a working period from 12:30 hours to 17:00 hours. This state can be recognized from real time beat to beat information from example by a state like HR>70 AND HRV>30. The number 44 indicates free time after work by a HR>90 and HRV>50. By testing the algorithm, the best mode of operation, the above HR is most likely to be HR long min or max and the above HRV is most likely to be HRV long. Within these activities illustrated by the above numbers 40 to 44 (for Monday per activity) in FIG. 26 specific colors per activity could be used to indicate the physical and emotional state in such a period as is indicated by the colour scheme in FIG. 23. A specific color could be used to represented stress beats. A specific color could be used to represented recovery beats. Such a stress beats, are part of an embodiment of this invention which comply to, for example, the following rule such as flow<HR*HRV=750 (low flow threshold). The number of stress beats and/or recovery beats could be counted for and recalculated to the total time of stress and/or recovery. In FIG. 26 another day is given such as Tuesday, so that the user is able to compare several days. Comparing Monday to Tuesday for example, makes it possible for the user to compare and analyze progress in physical, emotional state and planning's issues with respect to specific physical, emotional states, stress versus recovery, and activities. Another example of representation of the daily user state or representation of the hourly state of the user could be based upon FIG. 24 representation number 35. Every color of this indication 35 represents a user state duration which could be represented by a changing color optical density or visual (directed outwardly) quantity, percentage or size of that color (in this picture they all are represented for 100% as an example). Based upon a specific real time state for example HR & HRV combination (physical, emotional, or absolute emotional state) a specific music type or light type is selected. Two selection mechanisms could be used;

(1) selecting a music type or a light (scheme) type which fulfills an emotional match with the current real time state or

(2) selecting music type or light (scheme) type which steers the emotional state into direction of for example a better flow level or state. The real time selection relation between music type or light type and real time state can be derived based upon predefinition, personal selectable preferences, or big data analysis on what works for both mentioned two mechanisms. By this a new type of shuffler is part of an embodiment of this invention; the music type or light type is selected based upon the real time measured state of the user or group of users. Such an embodiment could be part of a dance or music system in which the music schemes and light schemes are selected based on multi-user heart rate information derive system, which are the base to program the dance music and dance light real time so that the group as a whole will go to a higher flow state or different state. Any further enhancements that combine real time hr and hrv state determination with real time information such as acceleration, speed, distance, or gps information is part of this invention.

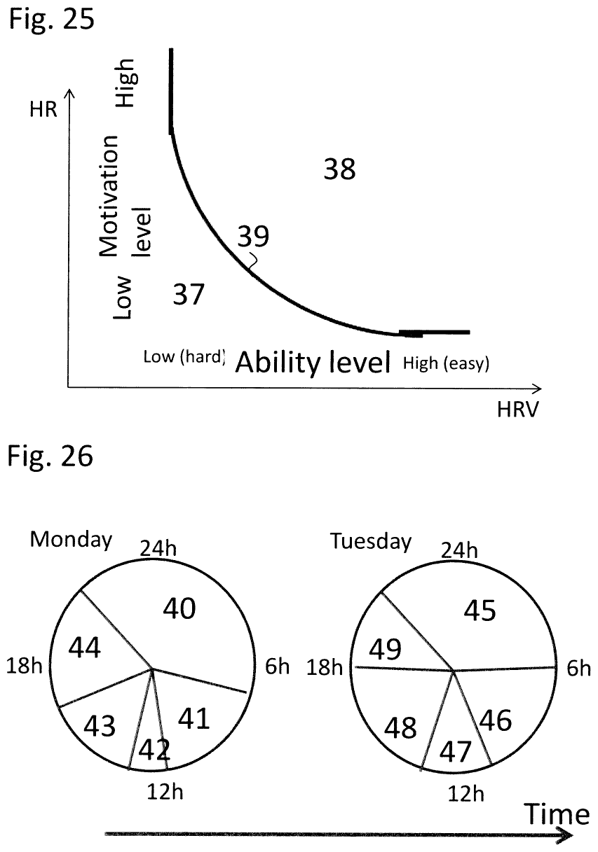

For user behavior change several factors are of importance; motivation, ability and triggers for change. In FIG. 25 is indicated a model for behavior change that is an embodiment of this invention by correlating motivation level of the user to the heart rate frequency level of the user, and by correlating the ability level of the user to the heart rate variability of the user. By also having a predetermined or adaptable trigger threshold per trigger type (in relation with HR and HRV, such as flow>3000) indicated by line 39 it is clear for which level of HR and HRV a trigger (such as advice or suggested action) is acceptable for the user due to his real time adaptability indicated by area 38 or is not successful (user is not real time adaptable or not flexible) indicated by area 37. By this way by measuring real time HR frequency and HRV value it is possible to organize triggers in time in such a way that the trigger is only activated or given when the trigger will lead to positive behavior change and success. Or that a trigger type is selected such that it will be successful for the real time HR and HRV (state) of the user at that moment. Embodiment of the invention is, in case of too low or too high flow value, that the user is advised and stimulated to do some specific exercise change to increase or decrease the flow fast depending on the activity. Examples of triggers are given by the following. For high HR and Low HRV (i.e. flow=HR short*HRV long=150*2) an example is “You are getting over trained; slow down and recover immediately”. For mid HR and Low HRV an example is “You could get over trained; slow down and recover”. For low HR and Low HRV an example is “You must recover faster; do some things that make you happy”. For high HR and mid HRV an example is “You should train on a lower pace”. For high HR and high HRV (i.e. flow=HR short*HRV long=130*80) an example is “You train on peak flow”. For mid HR and high HRV an example is “You could train on a higher pace”. For sport low HR and high HRV (i.e. flow=HR short*HRV long=95*100) an example is “You should train on a higher pace”. For low HR and mid HRV an example is “You could train on a higher pace”. For mid HR and mid HRV an example is “You are training correctly”.

Conventionally, people could recognize a stress indicator. An embodiment of this invention is that the flow algorithm is used to calculate a beat to beat real time stress indicator. Example of such an embodiment for such a real-time beat to beat stress indicator=750/(HR short*HRV long), or a different real-time beat to beat stress indicator=low threshold/(HR short*HRV long). Conventionally, people could recognize a maximum HR value (VCO2 max) for training as a conventional way of sporting. An embodiment of this invention is that the flow algorithm is used to calculate a beat to beat real time maximum HR value. By monitoring HRV long or flow smaller than a certain threshold for example HRV long<5 milliseconds or flow<120*5 milliseconds=600, and then log which HR max (HR CO2 max) corresponds to this moment in time. This HR max indicates the training moment real time for which it changes from aerobe to anaerobe training zone. By this the user can train in an optimized way, so that less recovery time is required and less muscular pain. A further embodiment of this invention is determining of a maximum CO2 heart rate during sporting or other intensive activity where the body uses such amount of O2 so that CO2 is produced in such quantities that the real time restoration of energy supply to the body is lacking. Then the maximal HR CO2 could be surpassed during the challenging tasks at hand. To warn for such a situation a realtime criteria is checked per heart beat. A best mode example for such a criteria is flow<500 (HR short*HRV long) or heart rate variability of HRV<5 msecs for HR>100. When this situation happens automatically the user is for example warned to slow down and take measures to keep under this HR CO2 max threshold. In this way the HR CO2 max could be found real time and the HR CO2 max is real time adapted during the exercises due to that new energy supply is regained or the user is recharged or new situation appears for which the user can strive for new challenges and new boundaries.

An embodiment of the invention is that the state algorithm recognizes in a real time the flow state level. The flow state level can be differentiated by recognizing different types of emotions for every flow state level. In the following list, the flow state level is correlated with the spectrum of emotions a human being may experience. The following flow states and corresponding emotions are sorted, from highest emotion frequency (energy state) to lowest emotion frequency state; joy & ecstasy (i.e. the values are given as an example and could be adapted to the individual flow>10000), gratitude (flow>9000), faith & trust (flow>8000), love (flow>7000), happiness (flow>6000), peace & serenity (flow>5000), willingness & cooperation & belonging (flow>4000), pride & fulfillment (flow>3500), self esteem, inspiration & creativity (flow>3400), passion & desire (flow>3300), courage (flow>3200), acceptance (flow>3100), neutrality & reasons & logic & security & non emotional response (flow>3000), vanity & envy & competitiveness & jealousy & judgment (flow>2500), fear & abandonment & insecurity & worry & shock (flow<2400), betrayal (flow<2200), anger & hate & malice (flow<1000), resentment & stubbornness & frustration (flow<900), dissatisfaction & unhappiness & boredom (flow<800), sorrow & grief & sadness & rejection & loneliness (flow<700), self-pity (flow<600), guilt & remorse & deception & dishonesty (flow<500), humiliation (flow<490), and shame & degradation (flow<480).

A further embodiment of this invention is a breathing guidance. Finding best inhale triggers or best exhale triggers via the heart rate information. An inventive way to do this is by using the realtime time between two beats and comparing this time with the average short term time between beats. When the realtime time between beats is longer than this average short term time between beats then it is time to exhale. And when the realtime time between beats is shorter than this average short term time between beats then it is time to inhale. By giving triggers to the user when to inhale or exhale the user is breathing in an enhanced way which promotes energy transport in the body in an effective and efficient manner. So that the flow state of the user will be enhanced in a short period of time. This can be done in groups of people, by synchronizing the inhale and exhale triggers finally the people get in an entranced group state.

A further embodiment of this invention is determining specific type of heart zones such as active, sport, passive, recovery, or stress beats based upon the state value. In this way the user can be informed on for example the percentages of the day that is executed in activities that are in sport state, active state or passive state. And how much time is spent in stress state or recovery state. When the state determination is also combined with a timing stamp, further big data analysis are possible. So it is interesting to log when these type of beats happen so that at a later moment it is possible to analyze the daily journal with respect to active, passive, sport, recovery and stress with respect to timing.

A further embodiment of this invention is determining the daily health score per time or time period based upon the state. An embodiment is that for example the number of recovery beats, active beats, and sport beats are added in such a way that the healthy beats are summed. A further enhancement is for example that the number of recovery beats are scored as very important beats and so are multiplied with a factor for example 10, for which the factor could be for example between 1 and 100. Such factors could also be used for the other type of beats.

Another type of beat is the stress beat. The number of stress beat could be subtracted from the daily health score as being a less healthy beat and so will have a negative impact on the daily health score. Also for the stress beat a factor could be used that is multiplied with the number of stress beats before it is subtracted from the daily health score. Such a daily health score could be summed over the day, or averaged over the time. The daily health score can be used for gamification between challenge and skill, or ability. By doing so the present invention provides methods that will, for example, feedback the daily health score in such a way that the user can easily understand that this day was stressful or relaxing day. Such a feedback is very interesting for example for the physician, coach, or user himself.

A further embodiment of this invention is inputting a forward direction state or ability the user want to do, by the user, coach or automatically by the system. By such an inputted forward directed state or ability, the system is inducing a trigger based upon the forward direction state such as “control” or ability like “presentation” and the real time state of the user. Such a trigger could be a course “presentation-level” based on the state the user is real time in. Another example is when the user is in a real time state “angry” the system could automatically select based upon the state “angry” relaxing music style and relaxing light color scheme such as green, or yellow coloring.

By doing so the present invention provides methods that will, for example, when the user indicates he want to recover by selecting the forward direction state for example “recover”, that the method selects a music type or a video type so that the user is able to relax faster.

A further embodiment of this invention is a personal power indicator. It indicates the stored human life energy in the body & mind still available for tasks or activities. It is an indication on the potential energy that can be derived from body & mind to use for tasks or activities. As such it is a useful indication to plan for the future to go in which direction based on energy available to spend. An example of personal power calculation method is the following: (low flow threshold+high flow threshold)*100%/2. Both thresholds are adaptive determined based upon the real time state. For example an alternative personal power is calculating average flow over a time.

A further embodiment of this invention is determining unconscious, conscious and super-conscious state based upon the state value. The determination of the unconscious, conscious, or super-conscious state delivers a variety in possibilities by selecting for example different feedback trigger types depending on in which state the user is in. For example in the super-conscious state the user is more able to make good decisions and so in this state the user is given trigger questions and so the user is able to make decisions based upon these super conscious trigger questions. An embodiment example of deriving these states is for unconscious” the flow is <30 (HR short*HRV long/25 for all three examples). For conscious: 30<conscious flow<100. And an example for super conscious flow>100. The type of trigger or type of advice depends on these different flow zone or states. For example for a flow<30; advice is given in line of more self aware triggers and/or repair action to induce very fast higher flow. For example for 30<flow<100; some motivational or directional question triggers appear. And for example for flow>100 gamifaction feedback is suggesting some high frequency emotional states the user is in or could strive for at that moment like bliss, peace, passion, or super bliss. The idea and concept here is that HR level represent game level in general. HRV level represents play level in general. So flow like real time HR*HRV, or any combination of real time HR and HRV is an indication for whether somebody can play a game at that level of flow. The state unconscious, conscious or super-conscious is a way to understand which type of game & play level the user is in and so which triggers the user is able to understand in the game. The ultimate play & game state will be super bliss and many people will strive for this situation. By means of determination of state type and via trigger type, directions can be given to go for example to a super bliss state, individually, or in a group of people.

A further embodiment of this invention is determining a decision risk, a decision loss chance, or a decision gain chance based upon the state. By doing so the present invention provides methods to indicate to the user based upon the real time state when to do (best) decision making. Based upon the real time state determination the decision risk, the decision loss chance and/or the decision gain chance can be derived and the user will appreciate to understand from his heart knowledge when to take good decisions and what risks are involved for that decision. An example of embodiment is a four quadrant decision making model depending on the real time HR and HRV value (flow or state on that moment). For high HR and low HRV values this model indicates that it will result in high loss chance and low gain chances when taking decisions. For high HR and high HRV in this example model it will result in low loss chances, and high gain chances when taking decisions. For low HR and low HRV in this model it will result in higher loss chances, and lower gain chances. For low HR and higher HRV in this model it will result in lower loss chances, and normal gain chances (but not high). Such a decision making model based on real time heart information is very useful for the user during taking decisions via real time feedback of his own heart information. Such a system will make decision making more easy and more based upon the mind & body information unconsciously, consciously, and super-consciously available. And when indicating that decisions making is not good the user is directed to a more flow energetic situation before taking any decisions.

A further embodiment of this invention is determining based upon subject state an advice or trigger for the user to optimize for a maximal performance. For example performance could be maximal for a flow between 50-1000 (HR short*HRV long/25). Above this performance level for example flow>1000 there is not enough positive stress to go for performance (it is just bliss). Below this for example flow<50 there is too much negative di-stress. So optimal stress is determined per real time beat information (best mode for example could be 50<flow<100) per heart beat. Another embodiment of state determination is in the scope of health care. Some health care problem areas can be solved more specifically with an embodiment of this invention. For example in the area of anaesthetic preoperative risk assessment, so called MET score. A MET score for pre anaesthetic energy is a risk indicator and the most important preoperative score for preoperative risk assessment on perioperative morbidity and mortality. If the amount of energy reserve for such a person is not high enough to encounter the operation then it is very likely that the patient is done harm.

To calculate such a risk it is categorized per type of surgery according to the energy needed to overcome the problem and the minimum required patient energy reserve. Up to now this MET score is provided by letting the patient filling in a questionnaire to obtain knowledge of what he or she can do, like walking, climbing stairs and biking. However there is currently no quantifying test other then the cardiologist treadmill. Another thing is that people consciously don't always know the answer because the people may never go biking. An embodiment of the invention is to use average flow state over time to measurement the energy reserve of such patients, by giving a task like climbing two stairs while wearing the heart rate sensor that measures the real time heart rate information and determines the health state via real time heart rate variability, flow or personal power. The exercise is done in a sport mode which is for heart rate a specific zone (for example HR>100 beats per minute & HR<180 beats per minute) and for heart rate variability a specific zone (for example HRV>1 milliseconds & HRV<10 milliseconds). There are for example two methods to do such a test:

1. Use a challenge level (for example climb two stairs or use treadmill with x watts resistance, or walk 1 km in x minutes), and the FLOW must stay above a certain threshold (for example flow>10; in this case flow=HR*HRV/25) and measure the real time HRV value.

2. The user is asked to undertake sport until the flow drops below a certain threshold. For example flow<10 and measure the challenge height done, like x stairs, x watts resistance during y time, or walked x km in y minutes.

Another embodiment of state determination in the scope of health care is about heart failure monitoring. A patient with a chronic disease has marginal life reserves (for example personal power). The disease can be better managed if chronic disease failure is recognized and treated in early onset. The real time flow determination can be a great help to manage heart failure patients: One way is by combining flow (real time HRV and HR) with an accelerometer or pedometer and a weighing machine. By monitoring real time flow and real time velocity (measurement) per distance an embodiment of the invention can indicate potential heart failure (medical treatment is necessary) when: Flow<80% of flow baseline, or delta flow decrease<30% of flow baseline within a sudden timeframe, or delta distance decrease<20% of the distance baseline, or weight increase>4% of weight baseline. Or. Flow has a sudden steep increase over time and HRV>heart failure threshold in milliseconds.

Above percentages are given as typical examples. A flow baseline can be determined a priory by big data analysis over a group of users, or specifically per user. Understanding flow can give patients insight in their disease and can provide healthy biofeedback improving their daily life.

Another embodiment of state determination in the scope of health care is about test ischemic heart diseases. Cardiac disease diagnosis is expensive. To differentiate from atypical complaints and truly ischemic heart disease treadmill testing or CT scan is done at the hospital. Flow measurement can provide a cheaper way to make ischemia more or less likely. A patient can be challenged on a treadmill together with real time measuring flow. If flow stays above 10 there is no reason to assume ischemia. If flow<10 there is a certain amount of stress and the treadmill challenge has to be stopped. Flow measurement that is combined with 4 patch 22 lead EKG measurement can instantly give the origin of ischemia including posterior. Readings can be sent to the cardiologist (in hospital). Such a practice can provide better diagnosis in heart diseases and will reduce cardiologic interventions and save costs. It can provide remote cardiologic diagnosis, and treatment without hospitalisation. Hospitalisations when diagnosis is early, can save direct and indirect healthcare costs, and provide better patient safety and empowerment. Another embodiment of state determination (in the scope of health care) is about sedation of patients undergoing treatment. Real time flow measurement can be used to monitor the state while patients are sedated. Patients undergoing treatment (like colposcopy) are getting anxiolytics to prevent possible anxiety or deeper sedation. The depth of sedation is difficult to manage. Patients undergoing treatment are anxious and could have low flow state. Flow can be a tool to find the right dept of sedation, because flow can be measured beat to beat and so professionally guide the sedation to sedate the patient to the right state of relaxation. For sedation a flow state for example at least 20 is strived for. Deeper sedation will give higher HRV and lower HR resulting in in a recovery state. While using pharmaceuticals (like dexmedetomidine) it is very important to use it in a reliable way during sedation. Real time flow measurement gives a feedback mechanism (whether it fulfils) as an anxiety measurement. Beat-to-beat flow measurement can provide in that. Another embodiment of state determination in the scope of health care is about COPD level determination. An inhale and exhale duration determination is done by determining a respiratory inhale duration when the real time milliseconds value between heart beats is smaller than the average milliseconds value between heart beats, and determining a respiratory exhale duration when the real time milliseconds value between heart beats is bigger than the average milliseconds value between heart beats. The level of lung efficiency of O2 and/or CO2 exchange is indicated by the duration time (or number of beats) of inhale and exhale by means of the short term HR increase time due to breath in (during coherence period, which is a period in time for which the long term HR is not changing more than 5 beats per minute) and the duration time (or number of beats) that the short term HR decrease due to breath out, takes during coherence period. And by using at least one of visual, sound, haptic or kinesthetic vibration it to provides the user breathing guidance to come into a more coherence state of breathing and so to minimize the COPD as much as possible. A higher level of O2 and/or CO2 efficiency exchange can be triggered by a deeper, longer period of inhale and exhale while the breathing is executed in phase with the heart coherence information the heart is providing.

A heart information sensor with specific requirements could be necessary in context of heart rate variability. For heart rate variability the precision of the measurement is of importance. So at least a measurement frequency of 100 HZ is likely to be reasonable for health care applications. For other applications for flow or heart rate variability less measurement frequency could be sufficient although not preferable. To solve the battery lifetime it would be very preferable to have a sensor that can select the measurement frequency in context of the requirements. Which could be for example that in normal mode the measurement frequency is 100 Hz to optimize for battery lifetime. And in case of emergency or any other additional precision is required in measurement of heart rate variability or flow, the measurement frequency is switched to a higher frequency than 100 Hz. For example sampling frequency of 200 Hz or even higher like 1000 Hz. This higher frequency of measurement of heart rate variability and so possible flow or state gives additional precision information and therefore so makes precise analyses in real time better (such as states of heart rate, respiratory rate, hear rate variability, stress, sleep, activity, flow). Such a frequency switching capable sensor is an embodiment of this invention.

Another embodiment of this invention is the use of a flow state determination for (flow) scoring information and (flow) filtering information as part of big data analysis. By this invention the subjects (human or animal) flow, or state determination can be determined per beat or per short period of time. By combining real-time state determination, during finding useful information (i.e. by googling) or filtering information (i.e. by indexing information), the information complying a higher level of flow indicates a better quality of that information for generating flow. To be able to do this, information first must be classified (and indexed) by the flow determination algorithm on the flow value that is correlated with this specific information source or trigger. So by monitoring the flow state of a subject during going through information or triggers the (real-time) flow state can be logged, in conjunction with the information source. An embodiment of such a system is for example that while somebody is browsing the internet the real-time measured flow state of this user is logged together with the internet information while browsing (the internet). In this way a database is filled with two mutual connected items; the information source and the resulting flow the information source has on the user. By doing this for groups of people browsing the internet, finally per information source, the multiple measured flow per user per information source is logged real-time. So then for example big data can be applied per information source with respect to the flow per individual, per group people, or per group type of people. Information sources can be any type of information source; internet webpage, radio station, television station, music type, person, animal, food type, scent type, or specific environment, etc. . . . This way it is easier to find per user or user types optimal sets of triggers to generate more flow for this user. By means of this embodiment per information source type, high flow stimulating triggers can be found, and also the low flow (stimulating) triggers can be found. Based upon for example these high flow and low flow triggers the user or coach can be advised to do certain things more often or restrict some triggers from the daily life to optimize for more flow.

Another embodiment of the invention is by using two heart rate sensor types in mutual dependency by switching seamlessly between different situations between these two sensor types in which a high accuracy ECG sensor is necessary for example during sporting and switching to a PPG sensor for situations in which a lower accuracy is good enough (normal working, home, or convenience situations). A problem of a PPG based heart rate sensor is, typically worn around the wrist, that such a PPG heart rate sensor accuracy is only accurate for low movement situations. For low movement situations the distance between sensor and skin is varying relative slowly during physical low impact situations. For physical high impact situations the PPG sensor will move in relation to the skin relative faster and in a less pre determinable way. By this the heart rate information of such a PPG sensor in the context of high physical impact movement cannot easily be compensated for the less accurate PPG heart information measurements. In such a high physical impact situation, due to the lower accuracy of the PPG sensor, it a better solution to use an ECG sensor for the heart rate information. An embodiment of the invention is to use the heart rate information as part of an accuracy heart information decision module to be able to suggest or automatically switch to the ECG sensor for situations in which the accuracy decision unit based upon the heart information decides that the PPG sensor has lower accuracy. Normally in rest the heart information indicates a working area of the heart in a specific way such as a heart rate zone or heart pulse pulse time in a zone representing a heart rate between of 30 beats per minute until 100 beats per minute. When the heart information is going outside the above mentioned range the accuracy of the PPG sensor information can be doubted and so a more accuracy sensor (more trustful sensor such as ECG) should be used.

Another embodiment of the invention is using an acceleration sensor as part of an accuracy heart information decision module to be able to suggest or automatically switch to the ECG sensor for situations in which the accuracy decision unit based upon the acceleration information that the PPG sensor has lower accuracy. For higher acceleration values the accuracy of the PPG sensor is not maintained and when the acceleration value trespasses a certain level another more accurate sensor type should be used. Or to use both the heart rate information and the acceleration information for determination of the accuracy of the heart beat information. The accuracy decision unit information can be displayed or informed in any way to the user.

Another embodiment of the invention is by using a flow algorithm feature “dynamic flow training” for optimising a maximal training effect. By indicating and instructing the user to increase a real-time flow level to a dynamic maximal flow level (a local maximum). When the real-time dynamic (over the top maximal) flow level starts going down then to instructing the user immediately to start recovery. By starting the recovery at the moment the real time (dynamic maximal) flow is decreasing, at the moment the flow starts to decline, the maximal training effect can be secured. This can also be used for activity scheduling. During warming up for a sporting activity, a minimum flow is required before a building up flow for the “dynamic flow training” can start. After the “dynamic flow training” phase, a flow recovery phase will be executed. A way to handle this is that the exercise challenge level should be made less when the flow maximal tops and will get less high, for example 40% below the maximal flow record for the current activity. Another way to handle this is that the exercise challenge should be made less when recovery time starts to surpass over a predefined threshold level (or a dynamic threshold level. Parameters such as recovery time threshold, or max (record) flow threshold can be tuned depending on personal status and sport type. Per activity type or sport type there could be a (number of max flow) scale. This scale is between a number of interval flow max and a number of endurance flow max which is differentiated by the number of flow peaks for a specific sport type, or training level on that scale. For example a running marathon could have the number of endurance flow max put on one. For example a soccer match could have the number of interval flow max put on for example 60. Such a max flow interval number per activity type can be made person dependent and/or sport type dependent and could be adaptable depending on the sport level of execution. By preselect a max flow interval number per activity type or user type or challenge level as a initial target for an activity and then based upon this target instruct the user for certain real time flow value target to act upon.

Although various preferred embodiments of the present invention have been disclosed for illustrative purposes, those skilled in the art will appreciate that various modifications, additions and/or substitutions are possible without departing from the scope and spirit of the present invention as disclosed in the claims.

1. Method for determining a subject state, comprising the steps of;
receiving a heartbeat information,
calculating a heart rate frequency value, wherein the heart rate frequency value is a number of heart beats per first time of the heart beat information,
calculating a heart rate variability value, wherein the heart rate variability value is a variation time per second time of the heart beat information,
determining the state value from the heart rate frequency value and the heart rate variability value by using one of multiplication, addition, or other type of relationship, wherein the first time and second time is one minute or less.

2. The method according claim 1, further wherein comprising the step of;
calculating the first heart rate variability value for 30 or less heart beats of the heart beat information, wherein the first heart rate variability value is an absolute difference between a mean RR time between heart beats and a RR time between heart beats,
determining the state value from the heart rate variability value.

3. The method according claim 2, further wherein comprising the step of;
calculating a second heart rate variability value for 60 or less heart beats, wherein the second heart rate variability value is calculated by taking an average of the first heart rate variability values.

4. The method according claim 1, further wherein comprising the step of;
determining the state value in a real time manner per heartbeat.

5-11. (canceled)

12. The method according claim 1, further comprising the step of sending at least one selected of the heart rate information, the heart rate variability, and the state, to a receiver of this information.

13. The method according claim 1, further comprising the step of determining the state value by comparing the state value with a state threshold value.

14. The method according claim 1, further comprising the step of determining an emotional state value by determining a change of the state value over a time with a state threshold value.

15. The method according claim 1, further wherein comprising the step of:
(i) determining a challenge level, or a skill level based upon the state value and a threshold state value and
(ii) determining subject advice or trigger based upon the determined challenge level and/or skill level.

16. The method according claim 1, further wherein comprising determining the state value comprises further determining based upon the state value, a flow state zone the user is in the flow state zone is at least selecting one of; angry, happy, sad, relaxed, excited, cheerful, serene, calm, fatigued, depressed, upset, stressed, and tense.

17. The method as in claim 1, further wherein comprising determining the flow state value comprises further determining based upon the flow state value, a training resistance value.

18. The method according claim 12, further comprising the steps of using at least one of;
client-server network, peer-to-peer networks, and block chain technology.

19. The method according claim 1, further comprising the step of determining whether the state value complies to at least one of; a minimum threshold or a maximum threshold, a state recovery change per time, which must be higher than a minimum change per time threshold, a state recovery time, which must be smaller than a maximum recovery time threshold, an unconscious state, conscious or a super-conscious state, a sedation state by a higher heart rate variability and lower heart rate frequency resulting in recovery state, and an ischemia state, which is smaller than an ischemia threshold.

20. The method according claim 1, comprising the step of sending at least one of the heart rate information, and determining the heart rate variability value while the subject is executing a challenge level and the subject is keeping the flow above a test flow level.

21. The method according claim 1, further comprising the steps of determining an executed challenge level of the subject until the state value is below a test flow level.

22. The method according claim 1, further comprising determining the state value, which comprises proposing an activity based upon a list of high flow state value activities.

23. The method according claim 13, further comprising the step of determining the state value comprises informing or advising or triggering the user if the state value is lower or higher than the state threshold value, wherein informing the user by means of at least one of; tactile, visual, auditory, smell and taste feedback.

24. The method according claim 14, further comprising the step of determining informing, advising or triggering based upon the state value or a total state value derived from the state value, based upon at least one from; an optimize peak flow, an optimizing for a maximal performance state, an average flow over time, a flow recovery time, a frequency, an intensity, a distance, a duration, a resistance, a stress state value by an inverse relation using the state value, and an activity.

25. Program code on a computer readable media, and such program code is loaded into a computer, by which computer is behaving as the method of claim 1.

26. A subject state determining device comprising at least one of smart watch, smart phone, heart rate information sensor, server machine, and cloud machine, such a device being configured to execute the method according to claim 1.

27. A subject state determining device comprising a heart rate information sensor providing heart beat information for method of claim 1, comprising a frequency switching mode to switch between a low frequency heart rate determination mode and a high frequency heart rate determination mode.
